(19) United States
(12) Patent Application Publication
(10) Pub. No.: US 2025/0129025 A1
(43) Pub. Date: Apr. 24, 2025

(54) METHODS OF TREATING PTSD AND NEUROLOGICAL DISORDERS

(71) Applicant: GATC HEALTH CORP, Irvine, CA (US)

(72) Inventors: Ian JENKINS, Cedar Hills, UT (US); Robert TINDER, Tacoma, WA (US); Vaishnavi NARAYAN, Lehi, UT (US); Noah TIPPETTS, Irvine, CA (US); Saman MIRZAEI, Irvine, CA (US); Patrick C. LILLEY, Irvine, CA (US); Gwendelyn C. LILLEY, Irvine, CA (US); Jayson UFFENS, Draper, UT (US)

(73) Assignee: GATC HEALTH CORP, Irvine, CA (US)

(21) Appl. No.: 18/718,380

(22) PCT Filed: Dec. 10, 2022
(86) PCT No.: PCT/US2022/081329

§ 371 Date: Jun. 10, 2024

Related U.S. Application Data
(60) Provisional application No. 63/288,365, filed on Dec. 10, 2021.

(51) Int. Cl. C07D 209/90; A61K 31/343; A61K 31/403; A61K 31/4045; A61K 31/4439; A61K 31/444; A61K 31/48; A61K 31/496; A61K 31/4985; A61K 31/5377; A61K 31/55; C07D 209/14; C07D 307/81; C07D 401/12; C07D 457/02; C07D 457/04; C07D 471/04; C07D 471/14; C07D 487/04

(52) U.S. Cl. CPC C07D 209/90; A61K 31/343; A61K 31/403; A61K 31/4045; A61K 31/4439; A61K 31/444; A61K 31/48; A61K 31/496; A61K 31/4985; A61K 31/5377; A61K 31/55; C07D 209/14; C07D 307/81; C07D 401/12; C07D 457/02; C07D 457/04; C07D 471/04; C07D 471/14; C07D 487/04

(57) ABSTRACT

Embodiments include therapeutic small molecules for use in treating addiction and neurological disorder by stimulating neuroplasticity. The therapeutic small molecule can increase neuroplasticity and improve neural function by affecting upstream regulators of FOS, JUN, BDNF, CDC42 and CCL2. The therapeutic small molecule can also aid in reduction of Amyloid plaques via binding with amyloid beta (a4).

### RELATED APPLICATIONS

This application claims priority to U.S. provisional patent application Ser. No. 63/288,365 filed on Dec. 10, 2021, the contents of which are incorporated herein by reference.

### REFERENCE TO SEQUENCE LISTING

The instant application contains a Sequence Listing which has been submitted electronically in ASCII format and is hereby incorporated by reference in its entirety.

### FIELD OF THE INVENTION

The invention relates to small molecules with therapeutic uses, specifically, it relates to a novel small molecule and methods to treat ailments such as a neurological disorder and addiction.

### BACKGROUND

Addiction can be defined as a biopsychosocial disorder characterized by compulsive engagement in rewarding stimuli despite adverse consequences. Addiction affects over 19.7 million people in the United States and presents a high financial and human toll. The total economic cost is greater than that of all types of diabetes and all cancers combined. These costs arise from, for example, the direct adverse effects of drugs and related healthcare costs, long-term complications, the loss of productivity and associated welfare costs, accidents, suicides, homicides and incarceration. To date there have been few effective pharmacological interventions and none that completely resolve the damage to the addicted brain.

Substance use disorder (SUD) can be defined as the persistent use of drugs despite substantial harm and adverse consequences. As an individual becomes increasingly dependent on a drug, discontinued use leads to withdrawal symptoms such as dysphoria. Substance use disorders are characterized by various mental/emotional, physical, and behavioral problems such as chronic guilt, an inability to reduce or stop consuming the substances despite repeated attempts, driving while intoxicated and physiological withdrawal symptoms. The development of dependence has been conceptualized as a neurobehavioral disorder that advances from impulsive drug use to compulsive drug abuse.

The term “behavioral addiction” can be defined as a compulsion to engage in a natural reward-which is a behavior that is inherently rewarding (i.e., desirable or appealing)-despite adverse consequences. Preclinical evidence has demonstrated that marked increases in the expression of ΔFosB through repetitive and excessive exposure to a natural reward induces the same behavioral effects and neuroplasticity as occurs in a drug addiction.

The brain disease model of addiction (BDMA) identifies addiction as a chronic relapsing brain disease suggesting that effective treatments to addiction include pharmaceutical and other medical interventions. BDMA holds that SUDs are chronic, relapsing brain diseases and that relapses are symptoms, and part of the expected course, of the disease. As with other diseases, SUDs can have multiple causes, including behavioral, environmental, and biological influences. Two particular proteins have been associated with addiction and SUD, ΔFosB and BDNF.

Protein fosB, also known as FosB and G0/G1 switch regulatory protein 3 (G0S3), is a protein that in humans is encoded by the FBJ murine osteosarcoma viral oncogene homolog B (FOSB) gene. The ΔFosB splice variant has been identified as playing a central, crucial (necessary and sufficient) role in the development and maintenance of addiction. ΔFosB overexpression (i.e., an abnormally and excessively high level of ΔFosB expression which produces a pronounced gene-related phenotype) triggers the development of addiction-related neuroplasticity throughout the reward system and produces a behavioral phenotype that is characteristic of an addiction.

Brain-derived neurotrophic factor (BDNF), or abrineurin, is a protein that, in humans, is encoded by the BDNF gene. BDNF acts on certain neurons of the central nervous system and the peripheral nervous system, helping to support survival of existing neurons, and encouraging growth and differentiation of new neurons and synapses.

Neuroplasticity, also known as neural plasticity, or brain plasticity, is the ability of neural networks in the brain to change through growth and reorganization. These changes range from individual neuron pathways making new connections, to systematic adjustments like cortical remapping. Examples of neuroplasticity include circuit and network changes that result from learning a new ability, environmental influences, practice and psychological stress.

Neuroplasticity was once thought to manifest only during childhood, but recent research has demonstrated that many aspects of the brain can be altered (i.e., are “plastic”) even through adulthood. However, the developing brain exhibits a higher degree of plasticity than the adult brain. Activity-dependent plasticity can have significant implications for healthy development, learning, memory and recovery from brain damage.

Based on recent studies, the regulation of gene expression has been proposed as a mechanism of drug addiction. Among other potential genes, induction or inhibition of FosB and/or BDNF have been proposed. However, direct interaction with FosB or BDNF has yet to yield significant improvement to the impaired function of the addicted brain. Studies have shown minor improvements with cannabinoid therapy, cranial stimulation and ketamine treatments. However, such improvements have not been widely accepted by the medical community.

Because conventional treatments for addiction are generally ineffective, there is a need for pharmaceuticals to treat addiction. Such treatments should have therapeutic effect through promoting neuroplasticity. Accordingly, embodiments of the invention include therapeutic peptides for treating addiction along with other disorders such as posttraumatic stress disorder (PTSD) and major depressive disorder (MDD).

### SUMMARY OF THE INVENTION

The inventions described and claimed herein have many attributes and embodiments including, but not limited to, those set forth or described or referenced in this brief summary. The inventions described and claimed herein are not limited to, or by, the features or embodiments identified in this summary, which is included for purposes of illustration only and not restriction.

Based on novel interpretations of molecular mechanisms obtained from biopsied brain tissue, Applicants have identified several underlying mechanisms for remodeling the addicted brain and resolving addiction. The technology has uncovered many signaling mechanisms that are promising in treating ailments such as posttraumatic stress disorder (PTSD) and major depressive disorder (MDD). In embodiments described herein, Applicants present therapeutic methods against particular targets to treat such ailments.

Accordingly, an object of the invention is to provide a novel compound that stimulates neuroplasticity. Another object of the invention is to provide a novel compound for treating a neurological disorder and/or addiction.

One embodiment is a method of treating a neurological disorder and/or addiction by stimulating neuroplasticity using a small molecule therapeutic.

One embodiment is a method of stimulating neuroplasticity using a small molecule therapeutic. Another embodiment is a method of preventing a neurological disorder and/or an addiction by stimulating neuroplasticity using a small molecule therapeutic

Applicants present a compound of Formula I, or an analog thereof.

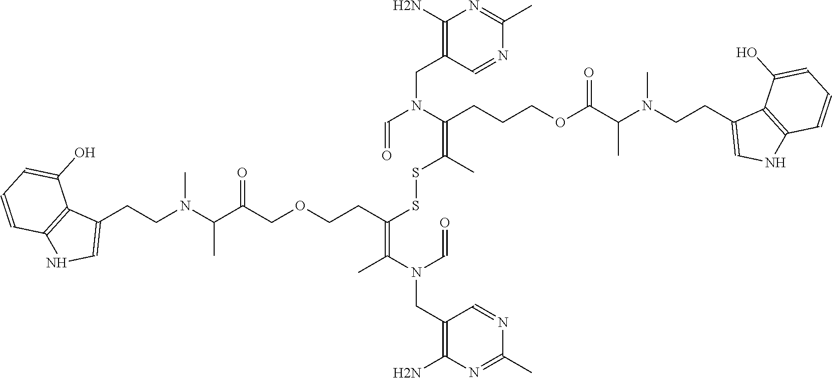 [SMILES: C/C(SS/C(CCOCC(=O)C(C)N(C)CCc1c[nH]c2cccc(O)c12)=C(/C)N(C=O)Cc1cnc(C)nc1N)=C(\CCCOC(=O)C(C)N(C)CCc1c[nH]c2cccc(O)c12)N(C=O)Cc1cnc(C)nc1N]

In another embodiment, the invention provides a compound, or a pharmaceutically acceptable salt thereof, wherein the compound is selected from A1, B1, C1, D1, E1, F1, G1, H1, A2, B2, C2, D2, E2, F2, G2, H2, A3, B3, C3, D3 and E3:

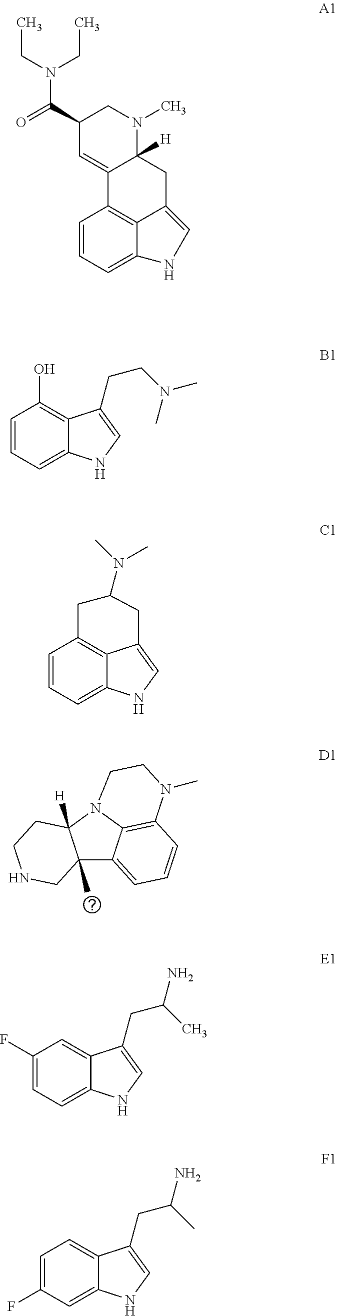 [SMILES: CCN(CC)C(=O)[C@@H]1C=C2c3cccc4[nH]cc(c34)C[C@H]2N(C)C1 ; CN(C)CCc1c[nH]c2cccc(O)c12 ; CN(C)C1Cc2cccc3[nH]cc(c23)C1 ; CN1CCN2c3c1cccc3[C@]1(C)CNCC[C@H]21 ; CC(N)Cc1c[nH]c2ccc(F)cc12 ; CC(N)Cc1c[nH]c2cc(F)ccc12]

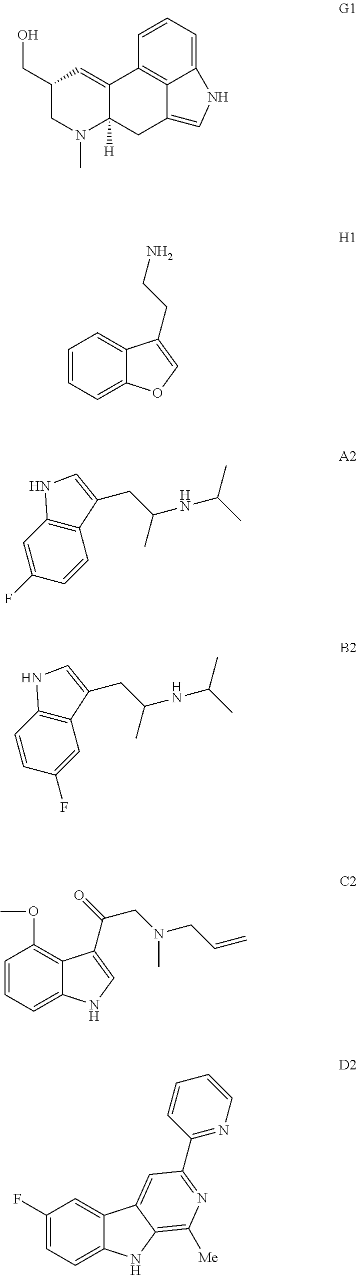

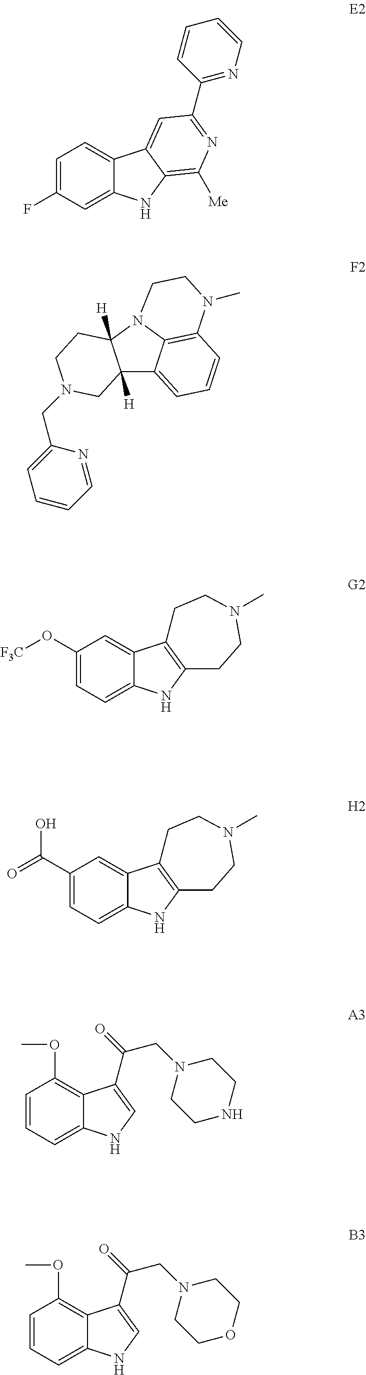

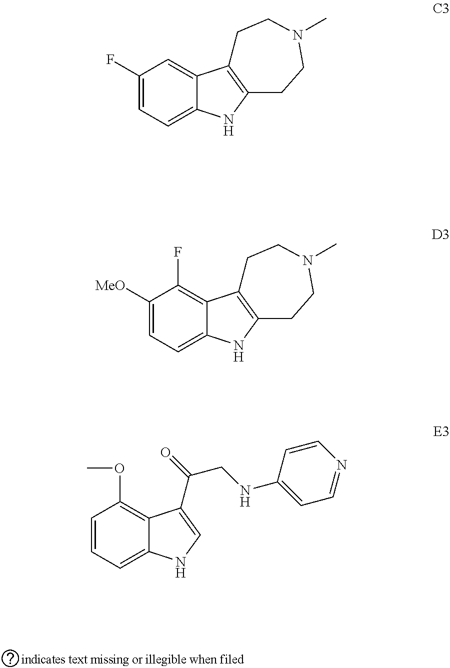

In one embodiment, the compounds described herein are useful in improving neuroplasticity. Another embodiment is a method of stimulating neuroplasticity in a subject wherein improving neuroplasticity prevents and/or treats an ailment such as a neurological disorder or addiction.

One embodiment is a method of treating a neurological disorder in a subject that includes administering a therapeutically effective amount of a compound of Formula I.

One embodiment is a method of treating a neurological disorder in a subject that includes administering a therapeutically effective amount of a compound selected from A1, B1, C1, D1, E1, F1, G1, H1, A2, B2, C2, D2, E2, F2, G2, H2, A3, B3, C3, D3 and E3.

One embodiment is a method for treating cognitive impairment, for delaying or slowing the progression of said cognitive impairment, or for reducing the rate of decline of cognitive function, in a subject having or at risk of having said cognitive impairment or decline of cognitive function, the method comprising administering a therapeutic amount of a compound disclosed herein.

One embodiment is a method of treating an ailment by administering a therapeutic amount of the compound of Formula I. The ailment can be, for example, anxiety, inflammation, addiction, post-traumatic stress disorder (PTSD), traumatic brain injury, depression, acute spinal cord injury, Alzheimer's disease, Amyotrophic Lateral Sclerosis (ALS), ataxia, Bell's Palsy, brain tumors, cerebral aneurysm, epilepsy and seizures, Guillain-Barre Syndrome, headache, head injury, hydrocephalus, meningitis, multiple sclerosis, muscular dystrophy, neurocutaneous syndromes, Parkinson's disease, stroke, headaches, encephalitis and myasthenia gravis.

One embodiment is a method of treating an ailment by administering a therapeutic amount of the compound of selected from A1, B1, C1, D1, E1, F1, G1, H1, A2, B2, C2, D2, E2, F2, G2, H2, A3, B3, C3, D3 and E3. The ailment can be, for example, inflammation, addiction, post-traumatic stress disorder (PTSD), traumatic brain injury, depression, acute spinal cord injury, Alzheimer's disease, Amyotrophic Lateral Sclerosis (ALS), ataxia, Bell's Palsy, brain tumors, cerebral aneurysm, epilepsy and seizures, Guillain-Barre Syndrome, headache, head injury, hydrocephalus, meningitis, multiple sclerosis, muscular dystrophy, neurocutaneous syndromes, Parkinson's disease, stroke, headaches, encephalitis and myasthenia gravis.

One embodiment is a method of treating addiction in a subject that includes administering a therapeutically effective amount of a compound of Formula I.

One embodiment is a method of treating addiction in a subject that includes administering a therapeutically effective amount of a compound selected from A1, B1, C1, D1, E1, F1, G1, H1, A2, B2, C2, D2, E2, F2, G2, H2, A3, B3, C3, D3 and E3. The addiction can be, for example, addiction to nicotine, cocaine, an opioid agonist or dependency on stimulants, nicotine, morphine, heroin, other opiates, amphetamines, cocaine, and/or alcohol.

One embodiment is a method of treating a neurological disorder comprising steps of (a) identifying a patient with a neurological disorder and (b) administering a therapeutic molecule to promote neuroplasticity. The therapeutic molecule can be the compound of Formula I or an analog of Formula I.

One embodiment is a method of treating a neurological disorder comprising steps of (a) identifying a patient with a neurological disorder and (b) administering a therapeutic molecule to promote neuroplasticity. The therapeutic molecule can be selected from A1, B1, C1, D1, E1, F1, G1, H1, A2, B2, C2, D2, E2, F2, G2, H2, A3, B3, C3, D3 and E3

One embodiment is a method of treating an addiction comprising steps of (a) identifying a patient with an addiction and (b) administering a therapeutic molecule to promote neuroplasticity. The therapeutic molecule can be the compound of Formula I or an analog of Formula I.

One embodiment is a method of treating an addiction comprising steps of (a) identifying a patient with an addiction and (b) administering a therapeutic molecule to promote neuroplasticity. The therapeutic molecule can be selected from A1, B1, C1, D1, E1, F1, G1, H1, A2, B2, C2, D2, E2, F2, G2, H2, A3, B3, C3, D3 and E3

One embodiment is a method of reversing the strident inhibition of dendritic arborization through rebalancing the expression ratio of FOSB, CDC42 and ENTPD4 by administering a therapeutically effective amount of a compound of Formula I.

One embodiment is a method of reversing the strident inhibition of dendritic arborization through rebalancing the expression ratio of FOSB, CDC42 and ENTPD4 by administering a therapeutically effective amount of a compound selected from A1, B1, C1, D1, E1, F1, G1, H1, A2, B2, C2, D2, E2, F2, G2, H2, A3, B3, C3, D3 and E3.

One embodiment is a method of restoring the impairment of thiamine metabolism by administering a therapeutically effective amount of a compound of Formula I.

One embodiment is a method of restoring the impairment of thiamine metabolism by administering a therapeutically effective amount of a compound selected from A1, B1, C1, D1, E1, F1, G1, H1, A2, B2, C2, D2, E2, F2, G2, H2, A3, B3, C3, D3 and E3.

One embodiment is a method of increasing presence of thiamine metabolism in the brain by administering a therapeutically effective amount of a compound of Formula I.

One embodiment is a method of increasing presence of thiamine metabolism in the brain by administering a therapeutically effective amount of a compound selected from A1, B1, C1, D1, E1, F1, G1, H1, A2, B2, C2, D2, E2, F2, G2, H2, A3, B3, C3, D3 and E3.

One embodiment is a method of improving neural metabolism by administering a therapeutically effective amount of a compound of Formula I.

One embodiment is a method of improving neural metabolism by administering a therapeutically effective amount of a compound selected from A1, B1, C1, D1, E1, F1, G1, H1, A2, B2, C2, D2, E2, F2, G2, H2, A3, B3, C3, D3 and E3.

One embodiment is a method of restoring neuronal excitation threshold by administering a therapeutically effective amount of a compound of Formula I.

One embodiment is a method of restoring neuronal excitation threshold by administering a therapeutically effective amount of a compound selected from A1, B1, C1, D1, E1, F1, G1, H1, A2, B2, C2, D2, E2, F2, G2, H2, A3, B3, C3, D3 and E3.

One embodiment is a method of reducing the relative abundance of CCL2 by administering a therapeutically effective amount of a compound of Formula I.

One embodiment is a method of reducing the relative abundance of CCL2 by administering a therapeutically effective amount of a compound selected from A1, B1, C1, D1, E1, F1, G1, H1, A2, B2, C2, D2, E2, F2, G2, H2, A3, B3, C3, D3 and E3.

One embodiment is a method of reducing inflammation via reduction of CCL2 by administering a therapeutically effective amount of a compound of Formula I.

One embodiment is a method of reducing inflammation via reduction of CCL2 by administering a therapeutically effective amount of a compound selected from A1, B1, C1, D1, E1, F1, G1, H1, A2, B2, C2, D2, E2, F2, G2, H2, A3, B3, C3, D3 and E3.

One embodiment is a method of improving neural function by administering a therapeutically effective amount of a compound of Formula I.

One embodiment is a method of improving neural function by administering a therapeutically effective amount of a compound selected from A1, B1, C1, D1, E1, F1, G1, H1, A2, B2, C2, D2, E2, F2, G2, H2, A3, B3, C3, D3 and E3.

One embodiment is a method of reducing of amyloid plaques via binding with amyloid beta (a4) by administering a therapeutically effective amount of a compound of Formula I or an analog of Formula I. Accordingly, another embodiment is a method of treating Parkinson's Disease by administering a therapeutically effective amount of a compound of Formula I or an analog of Formula I.

One embodiment is a method of reducing of amyloid plaques via binding with amyloid beta (a4) by administering a therapeutically effective amount of a compound of Formula I or an analog of Formula I. Accordingly, another embodiment is a method of treating Parkinson's Disease by administering a therapeutically effective amount of a compound selected from A1, B1, C1, D1, E1, F1, G1, H1, A2, B2, C2, D2, E2, F2, G2, H2, A3, B3, C3, D3 and E3.

In one embodiment, the effective dose of a therapeutic compound disclosed herein is about 4 milligrams (mg). In one embodiment, the upper limit of safety is about 1.2 grams. In one embodiment, the dose range for clinical administration is about 4 mg to 80 mg.

In still another aspect, the present specification provides a use of the therapeutic small molecule or the pharmaceutical composition including the same in the preparation of drugs for the prevention or treatment of a neurological disorder or addiction.

Other features and advantages of aspects of the present invention will become apparent from the following more detailed description, taken in conjunction with the accompanying drawings, which illustrate, by way of example, the principles of aspects of the invention.

### BRIEF DESCRIPTION OF THE DRAWINGS

The accompanying drawings illustrate aspects of the present invention. In such drawings:

FIG. 1 depicts the chemical structure of the compound of Formula I (“Somahol”) with numbered atoms.

FIG. 2 depicts the oxytocin signaling pathway.

FIG. 3 depicts molecular signaling pathways regulated by the 5-HT7 receptor.

FIG. 4 depicts RNASE I interacting with adjacent genes.

FIG. 5 depicts CCL2 interacting with adjacent genes.

FIG. 6 depicts FOSB interacting with adjacent genes.

FIG. 7 depicts SLC39A8 interacting with adjacent genes.

FIG. 8 depicts C9ORF135 interacting with adjacent genes.

FIG. 9 depicts ENTPD4 interacting with adjacent genes.

FIG. 10 depicts a scaffold molecule. In embodiments, the hydroxyl and/or carbonyl groups can be replaced with another functional group.

FIG. 11 is a graph of the results of a 5-HT2A Human Serotonin GPCR Cell Based Agonist Arrestin Assay.

FIG. 12 is a graph of the results of a 5-HT6 Human Serotonin GPCR Cell Based Agonist Arrestin Assay.

FIG. 13 is a graph of the results of a TRKB Human RTK Kinase Cell Based Agonist Functional Assay.

FIG. 14 is a graph of the results of a 5-HT7 Human Serotonin GPCR Cell Based Agonist CAMP Assay.

FIG. 15 is a graph of the results of a 5-HT2B Human Serotonin GPCR Cell Based Agonist Calcium Flux Assay.

FIG. 16 is a table of the results of agonist assays from each molecule: A1, B1, C1, D1, E1, F1, G1, H1, A2, B2, C2, D2, E2, F2, G2, H2, A3, B3, C3, D3 and E3.

### DEFINITIONS

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can be in certain instances be used interchangeably.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

As applicable, the terms “about” or “generally”, as used herein in the specification and appended claims, and unless otherwise indicated, means a margin of +/−20%. Also, as applicable, the term “substantially” as used herein in the specification and appended claims, unless otherwise indicated, means a margin of +/−10%. It is to be appreciated that not all uses of the above terms are quantifiable such that the referenced ranges can be applied.

The term “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An active agent can be a primary active agent, or in other words, the component(s) of a composition to which the whole or part of the effect of the composition is attributed. An active agent can be a secondary agent, or in other words, the component(s) of a composition to which an additional part and/or other effect of the composition is attributed. A “pharmaceutical composition” can include the combination of an active agent, such as a therapeutic peptide, with a carrier, inert or active, in a sterile composition suitable for diagnostic or therapeutic use in vitro, in vivo or ex vivo.

The term “analog” or “chemical analog” refers to a compound having a structure similar to that of another compound (i.e., Somahol), but differing from it in respect to a certain component. It can differ in one or more atoms, functional groups, or substructures, which are replaced with other atoms, groups, or substructures.

The term “alkyl” as employed herein alone or as part of another group designates both straight- and branched-chain saturated hydrocarbons containing 1 to 20 carbons. Examples of alkyl groups are methyl, ethyl, n-propyl, isopropyl, n-butyl, isobutyl, tert-butyl, pentyl, hexyl, isohexyl, heptyl, 4,4-dimethylpentyl, octyl, 2,2,4-trimethylpentyl, nonyl, decyl, undecyl, dodecyl, and the various branched-chain isomers thereof.

The term “alkylene” as employed herein alone or as part of another group refers to a divalent radical of an alkyl group as described above, containing 1 to 20 carbons. Examples of alkylene groups are methylene, ethylene, trimethylene, tetramethylene, pentamethylene, hexamethylene, heptamethylene, octamethylene, and the various branched-chain isomers thereof.

The term “alkenyl” as employed herein alone or as part of another group includes both straight- and branched-chain hydrocarbons containing 2 to 20 carbons, preferably 2 to 12 carbons, and more preferably 2 to 8 carbons, and includes 1 to 6 double bonds. Examples of alkenyl groups are vinyl, 2-propenyl, 2-butenyl, 3-butenyl, 3-pentenyl, 4-pentenyl, 2-hexenyl, 3-hexenyl, 2-heptenyl, 4-heptenyl, 3-octenyl, 3-nonenyl, 4-decenyl, 3-undecenyl, and 4,8,12-tetradecatrienyl.

The term “alkynyl” as employed herein alone or as part of another group includes both straight- and branched-chain hydrocarbons containing from 2 to carbons, preferably 2 to 12 carbons, and more preferably 2 to 8 carbons, and includes 1 to 6 triple bonds and optionally 1 to 3 double bonds. Examples of alkynyl groups are 2-propynyl, 3-butynyl, 4-pentynyl, 2-hexynyl, 4-heptynyl, 3-octynyl, 3-nonynyl, 4-decynyl, 3-undecynyl and 4-dodecynyl.

The term “alkanoyl” or “acyl” as indistinctively used herein alone or as part of another group refers to an alkyl group attached to a carbonyl group. In the context of the present invention, the terms “alkanoyl” and “acyl” have the same meaning. Thus, a Cn alkanoyl or acyl groups is a Cn-1 alkyl group attached to a carbonyl group. Examples of alkanoyl or acyl groups are acetyl, propionyl and butyroyl.

The term “alkoxy” as employed herein alone or as part of another group designates an alkyl group containing 1 to 20 carbons, preferably 1 to 10 carbons, and more preferably 1 to 8 carbons linked to an oxygen atom. Examples of alkoxy groups are methoxy, ethoxy, n-propoxy, isopropoxy, n-butoxy, isobutoxy, tert-butoxy, pentoxy, hexyloxy, isohexyloxy, heptyloxy, 4,4-dimethylpentoxy, octyloxy, 2,2,4-trimethylpentoxy, nonyloxy, decyloxyl, undecyloxy, dodecyloxy, and the various branched-chain isomers thereof.

The term “cycloalkyl” as employed herein alone or as part of another group includes saturated cyclic hydrocarbon groups containing 1 to 3 rings, including monocyclic alkyl, bicyclic alkyl and tricyclic alkyl systems and containing a total of 3 to 20 carbon atoms, preferably 3 to 10 carbons, forming part of the ring system. Examples of cycloalkyl groups are cyclopropyl, cyclobutyl, cyclopentyl, cyclohexyl, cycloheptyl, cyclooctyl, cyclodecyl, cyclododecyl, adamantyl and bicyclo[3.3.3]undecanyl.

The term “cycloalkenyl” as employed herein alone or as part of another group includes partially unsaturated cyclic hydrocarbon groups containing 1 or 2 double bonds and having 1 to 3 rings, including monocyclic alkyl, bicyclic alkyl and tricyclic alkyl systems, containing a total of 4 to 12 carbons, preferably 5 to carbons, as part of the ring system. Examples of cycloalkenyl groups include, but are not limited to, cyclobutenyl, cyclopentenyl, cyclohexenyl, cycloheptenyl, cyclooctenyl, cyclohexanediyl and cycloheptanediyl.

The term “aryl” as employed herein alone or as part of another group refers to monocyclic or bicyclic aromatic groups containing 6 to 10 carbons in the ring portion, such as phenyl or naphthyl (including 1-naphthyl and 2-naphthyl) and may optionally include 1 to 3 additional fused carbocyclic rings, such as cycloalkyl. Examples of aryl groups are phenyl, 1-naphthyl, 2-naphthyl and tetrahydronaphthyl.

The term “heterocyclyl” as employed herein alone or as part of another group refers to a 5-, 6-, or 7-membered saturated or partially unsaturated ring which includes 1 to 2 heteroatoms, selected from the group consisting of nitrogen, oxygen and/or sulfur, and such rings optionally fused to an aryl, cycloalkyl, heteroaryl or heterocyclyl ring. The heterocyclyl group is linked through a carbon atom or a heteroatom.

The term “heteroaryl” as employed herein alone or as part of another group refers to a 5- or 6-membered aromatic ring which includes 1, 2, 3 or 4 heteroatoms, selected from the groups consisting of nitrogen, oxygen or sulfur, and such rings optionally fused to an aryl, cycloalkyl, heteroaryl or heterocyclyl ring. When an heteroaryl group is fused to and aryl it is called a “bicyclic heteroaryl”.

The term “glycosyl” as employed herein refers to 1-O-β-D-galactopyranosyl (galactose), 1-O-β-D-glucopyranosyl (glucose) and 1-O-α-D-glucopyranosyl-α-D-glucopyranosyl (trehalose).

The term “functional partner” refers to one or more compounds (e.g., genes or proteins) that act in a system to support a function. A functional partner can act through one or more intermediates (e.g., genes, small molecules or proteins).

The term “dendrite” refers to a branched protoplasmic extension of a nerve cell that propagates the electrochemical stimulation received from other neural cells to the cell body, or soma of the neuron from which the dendrite projects. Electrical stimulation is transmitted onto dendrites by upstream neurons (usually via their axons) via synapses which are located at various points throughout the dendritic tree. Dendrites appear to be capable of plastic changes during the adult life of animals, including invertebrates. Neuronal dendrites have various compartments known as functional units that are able to compute incoming stimuli. These functional units are involved in processing input and are composed of the subdomains of dendrites such as spines, branches, or groupings of branches. Therefore, plasticity that leads to changes in the dendrite structure will affect communication and processing in the cell. During development, dendrite morphology is shaped by intrinsic programs within the cell's genome and extrinsic factors such as signals from other cells. But in adult life, extrinsic signals become more influential and cause more significant changes in dendrite structure compared to intrinsic signals during development.

The term “post-traumatic stress disorder” or “PTSD” refers to a mental and behavioral disorder that can develop because of exposure to a traumatic event, such as sexual assault, warfare, traffic collisions, violence, etc. Symptoms can include disturbing thoughts, feelings, or dreams related to the events, mental or physical distress to trauma-related cues, attempts to avoid trauma-related cues, alterations in the way a person thinks and feels, and an increase in the fight-or-flight response. PTSD causes biochemical changes in the brain and body that differ from other psychiatric disorders such as major depression.

The term “traumatic brain injury” or “TBI” refers to an injury to the brain caused by an external force. TBI can be classified based on severity (ranging from mild traumatic brain injury [mTBI/concussion] to severe traumatic brain injury), mechanism (closed or penetrating head injury), or other features (e.g., occurring in a specific location or over a widespread area). TBI can lead to temporary or permanent impairment of cognitive, physical and psychosocial functions. TBI is not a single pathophysiological event but a complex disease process, and causes structural damage and functional deficits that are due to both primary and secondary injury mechanisms. The primary injury is the result of the immediate mechanical disruption of brain tissue that occurs at the time of exposure to the external force and includes contusion, damage to blood vessels (hemorrhage), and axonal shearing, in which the axons of neurons are stretched and torn. Secondary injury evolves over minutes to months after the primary injury, and is the result of cascades of metabolic, cellular and molecular events that ultimately lead to brain cell death, tissue damage and atrophy.

The term “alcohol dependency” refers to a chronic medical condition that typically includes a current or past history of excessive drinking, a strong craving for alcohol, continued use despite repeated problems with drinking and an inability to control alcohol consumption.

The term “multiple sclerosis” or “MS” refers to an inflammatory demyelinating disease of the central nervous system (CNS) which involves a complex interaction between immune system and neural cells.

The term “neurological disorder” broadly refers to a disorder of the nervous system. Neurological disorders can affect the brain as well as the nerves found throughout the human body and the spinal cord. Structural, biochemical or electrical abnormalities in the brain, spinal cord or other nerves can result in a range of symptoms. Neurological disorders include, for example, acute spinal cord injury, Alzheimer's disease, Amyotrophic Lateral Sclerosis (ALS), ataxia, Bell's Palsy, brain tumors, cerebral aneurysm, epilepsy and seizures, Guillain-Barré Syndrome, headache, head injury, hydrocephalus, meningitis, multiple sclerosis, muscular dystrophy, neurocutaneous syndromes, Parkinson's disease, stroke, headaches, encephalitis and myasthenia gravis.

The term “transcriptional regulation” refers to the means by which a cell regulates the conversion of DNA to RNA (transcription), thereby orchestrating gene activity. A single gene can be regulated in a range of ways, from altering the number of copies of RNA that are transcribed, to the temporal control of when the gene is transcribed. This control allows the cell or organism to respond to a variety of intra- and extracellular signals and thus mount a response. Transcription factors are proteins that bind to specific DNA sequences in order to regulate the expression of a given gene.

The term “oxytocin” or “OT” refers to a nonapeptide synthesized by the magno-cellular neurons located in the supraoptic (SON) and paraventricular (PVN) nuclei of the hypothalamus. It exerts a wide variety of central and peripheral effects. However, its best-known and most well-established roles are stimulation of uterine contractions during parturition and milk release during lactation. Oxytocin also influences cardiovascular regulation and various social behaviors. The actions of OT are all mediated by one type of OT receptor (OTR). This is a transmembrane receptor belonging to the G-protein-coupled receptor superfamily. The main signaling pathway is the Gq/PLC/Ins3 pathway, but the MAPK and the RhoA/Rho kinase pathways are also activated, contributing to increased prostaglandin production and direct contractile effect on myometrial cells.

The term “protein fosB,” “FosB,” “G0/G1 switch regulatory protein 3” or “GOS3” refers to a protein that in humans is encoded by the FBJ murine osteosarcoma viral oncogene homolog B (FOSB) gene. The FOS gene family includes four members: FOS, FOSB, FOSL1, and FOSL2. These genes encode leucine zipper proteins that can dimerize with proteins of the JUN family (e.g., c-Jun, JunD), thereby forming the transcription factor complex AP-1. As such, the FOS proteins have been implicated as regulators of cell proliferation, differentiation, and transformation.

The term “brain-derived neurotrophic factor,” “BDNF,” or “abrineurin” refers to a protein that, in humans, is encoded by the BDNF gene. BDNF is a member of the neurotrophin family of growth factors, which are related to the canonical nerve growth factor. Neurotrophic factors are found in the brain and the periphery. FOS, JUN, BDNF, CDC42 and CCL2 via cohort activity of upstream interaction. The Fos gene family includes four members: FOS, FOSB, FOSL1, and FOSL2. These genes encode leucine zipper proteins that can dimerize with proteins of the JUN family, thereby forming the transcription factor complex AP-1. As such, the FOS proteins have been implicated as regulators of cell proliferation, differentiation, and transformation. In some cases, expression of the FOS gene has also been associated with apoptotic cell death.

The term “c-Jun” refers to a protein that in humans is encoded by the JUN gene. c-Jun, in combination with c-Fos, forms the AP-1 early response transcription factor. It was first identified as the Fos-binding protein p39 and only later rediscovered as the product of the JUN gene. c-Jun was the first oncogenic transcription factor discovered. The proto-oncogene c-Jun is the cellular homolog of the viral oncoprotein v-Jun (P05411). The human JUN encodes a protein that is highly similar to the viral protein, which interacts directly with specific target DNA sequences to regulate gene expression.

The term “brain-derived neurotrophic factor,” “BDNF,” or “abrineurin” refers to a protein that, in humans, is encoded by the BDNF gene. BDNF is a member of the neurotrophin family of growth factors, which are related to the canonical nerve growth factor.

The term “CDC42” or “cell division control protein 42 homolog” refers to a protein involved in regulation of the cell cycle. Human Cdc42 is a small GTPase of the Rho family, which regulates signaling pathways that control diverse cellular functions including cell morphology, cell migration, endocytosis and cell cycle progression.

The term “CCL2,” “chemokine (C-C motif) ligand 2” or “monocyte chemoattractant protein 1 (MCP1)” or “small inducible cytokine A2” refers to a small cytokine that belongs to the CC chemokine family. CCL2 recruits monocytes, memory T cells, and dendritic cells to the sites of inflammation produced by either tissue injury or infection. CCL2 is implicated in pathogeneses of several diseases characterized by monocytic infiltrates, such as psoriasis, rheumatoid arthritis and atherosclerosis.

Neuroplasticity, also known as neural plasticity, or brain plasticity, is the ability of neural networks in the brain to change through growth and reorganization. These changes range from individual neuron pathways making new connections, to systematic adjustments like cortical remapping. Examples of neuroplasticity include circuit and network changes that result from learning a new ability, environmental influences, practice, and psychological stress. Activity-dependent plasticity can have significant implications for healthy development, learning, memory and recovery from brain damage.

The term “STRING” refers to a graphical depiction of relationships between genes or proteins. Functional links between proteins can often be inferred from genomic associations between the genes that encode them: groups of genes that are required for the same function tend to show similar species coverage, are often located in close proximity on the genome (in prokaryotes), and tend to be involved in gene-fusion events. The database STRING is a precomputed global resource for the exploration and analysis of these associations. Because the three types of evidence differ conceptually, and the number of predicted interactions is very large, it is essential to be able to assess and compare the significance of individual predictions. Thus, STRING contains a unique scoring-framework based on benchmarks of the different types of associations against a common reference set, integrated in a single confidence score per prediction. The graphical representation of the network of inferred, weighted protein interactions provides a high-level view of functional linkage, facilitating the analysis of modularity in biological processes.

The term “dendritic arborization” or “dendritic branching” refers to a multi-step biological process by which neurons form new dendritic trees and branches to create new synapses. This ability for dendritic growth is thought to play a role in learning and memory formation.

The term “thiamine” or “vitamin B1” refers to an essential nutrient that serves as a cofactor for a number of enzymes, mostly with mitochondrial localization. Some thiamine-dependent enzymes are involved in energy metabolism and biosynthesis of nucleic acids whereas others are part of the antioxidant machinery. The brain is highly vulnerable to thiamine deficiency due to its heavy reliance on mitochondrial ATP production. This is more evident during rapid growth (i.e., perinatal periods and children) in which thiamine deficiency is commonly associated with either malnutrition or genetic defects. Thiamine deficiency contributes to a number of conditions spanning from mild neurological and psychiatric symptoms (confusion, reduced memory, and sleep disturbances) to severe encephalopathy, ataxia, congestive heart failure, muscle atrophy and even death.

The term “subject” or “patient” refers to any single animal, more preferably a mammal (including such non-human animals as, for example, dogs, cats, horses, rabbits, zoo animals, cows, pigs, sheep, and non-human primates) for which treatment is desired. Most preferably, the patient herein is a human.

The term “pharmaceutically acceptable carrier” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutically acceptable composition” as used herein refers to a composition comprising at least one compound as disclosed herein formulated together with one or more pharmaceutically acceptable carriers.

As used herein, the term “prevention” means all of the actions by which the occurrence of the disease is restrained or retarded.

The term “treating” or “treatment” refers to one or more of (1) inhibiting the disease (i.e., arresting further development of the pathology and/or symptomatology); and (2) ameliorating the disease (i.e., reversing the pathology and/or symptomatology) such as decreasing the severity of disease.

The term “administration” refers to the introduction of an amount of a predetermined substance into a patient by a certain suitable method. The composition disclosed herein may be administered via any of the common routes, as long as it is able to reach a desired tissue, for example, but is not limited to, inhaling, intraperitoneal, intravenous, intramuscular, subcutaneous, intradermal, oral, topical, intranasal, intrapulmonary, or intrarectal administration.

All numerical designations, e.g., pH, temperature, time, concentration, and molecular weight, including ranges, are to be understood as approximations in accordance with common practice in the art. When used herein, the term “about” may connote variation (+) or (−) 1%, 5% or 10% of the stated amount, as appropriate given the context. It is to be understood, although not always explicitly stated, that the reagents described herein are merely exemplary and that equivalents of such are known in the art.

Many known and useful compounds and the like can be found in Remington's Pharmaceutical Sciences (13th Ed), Mack Publishing Company, Easton, PA—a standard reference for various types of administration. As used herein, the term “formulation(s)” means a combination of at least one active ingredient with one or more other ingredient, also commonly referred to as excipients, which may be independently active or inactive. The term “formulation” may or may not refer to a pharmaceutically acceptable composition for administration to humans or animals and may include compositions that are useful intermediates for storage or research purposes.

Other technical terms used herein have their ordinary meaning in the art that they are used, as exemplified by a variety of technical dictionaries. The particular values and configurations discussed in these non-limiting examples can be varied and are cited merely to illustrate at least one embodiment and are not intended to limit the scope thereof.

### Detailed Description

It is to be understood that both the foregoing general description and the following detailed description are exemplary and explanatory and are intended to provide further explanation of the subject technology as claimed. Additional features and advantages of the subject technology are set forth in the description below, and in part will be apparent from the description, or may be learned by practice of the subject technology. The advantages of the subject technology will be realized and attained by the structure particularly pointed out in the written description and claims hereof.

### Somahal

One embodiment is a compound of Formula I, or an analog thereof (also referred to as “Somahol”), a compound developed by analysis of a combination of multi-omic and bioinformatic tools including protein to protein interactions, convolutional neural networks, pathway modeling and target receptor site activation studies.

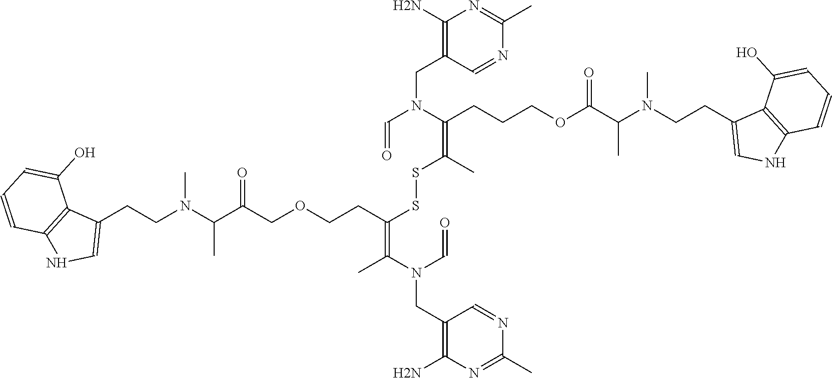 [SMILES: C/C(SS/C(CCOCC(=O)C(C)N(C)CCc1c[nH]c2cccc(O)c12)=C(/C)N(C=O)Cc1cnc(C)nc1N)=C(\CCCOC(=O)C(C)N(C)CCc1c[nH]c2cccc(O)c12)N(C=O)Cc1cnc(C)nc1N]

*IUPAC name: (4Z)-4-{N-[(4-amino-2-methylpyrimidin-5-yl)methyl]formamido}-5-{[(2Z)-3-{N-[(4-amino-2-methylpyrimidin-5-yl)methyl]formamido}-6-[(2-{[2-(4-hydroxy-1H-indol-3-yl)ethyl](methyl)amino}propanoyl)oxy]hex-2-en-2-yl]disulfanyl}hex-4-en-1-yl 2-{[2-(4-hydroxy-1H-indol-3-yl)ethyl](methyl)amino}propanoate)

### Structure and Synthesis

Embodiments include methods of synthesis of Somahal as well as analogs of Somahol. Embodiments include methods of synthesis of a compound of formula A1, B1, C1, D1, E1, F1, G1, H1, A2, B2, C2, D2, E2, F2, G2, H2, A3, B3, C3, D3 and E3 as well as analogs thereof.

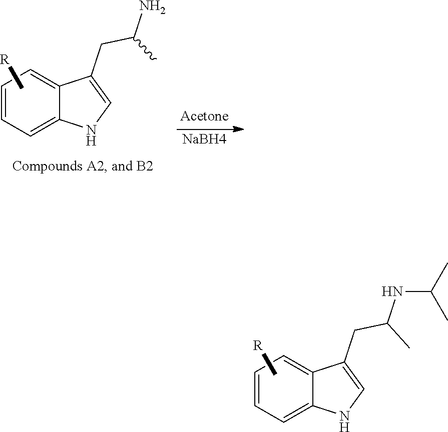

### Compounds A2 and B2

25 mg of indole was charged to a 14 mL open test tube along with 3 mL of Toluene and 1 mL of Acetone and 20 μL of Acetic Acid. The reaction mixture was heated to reflux temperature for 30 minutes. After 30 minutes the reaction mixture was allowed to cool and 25 mg of Sodium Borohydride was added as a solid and the mixture stirred for 1 hour. 5 mL of water was added and the reaction mixture extracted. The top layer was collected and dried with Sodium Sulfate, followed by concentration in vacuo to a brown oil which was purified by silica gel chromatography. (Isco Combiflash, 12 g. Redisep Gold, Ethyl Acetate, Heptane gradient, 0-100% ethyl acetate over 15 minutes) to afford 21 mg of the desired isopropylated amine product.

The product fractions were then combined and concentrated in vacuo (SpeedVac) and the resulting oils were used in screening.

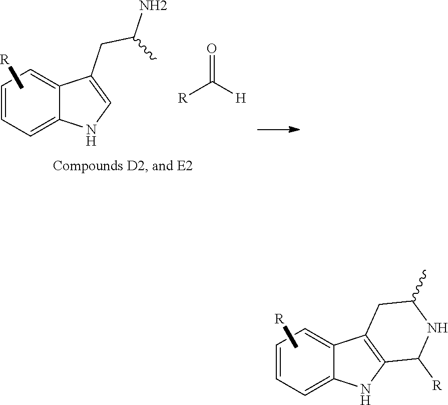

### Compounds D2 and E2

25 mg of the indol was charged to a 14 mL open test tube along with 3 mL of Toluene and 1 mL of aldehyde and 20 μL of Acetic Acid. The reaction mixture was heated to reflux temperature for 30 minutes. After 30 minutes the reaction mixture was allowed to cool. 5 ml of water was added and the reaction mixture extracted. The top layer was collected and dried with Sodium Sulfate, followed by concentration in vacuo to a brown oil which was purified by silica gel chromatography. (Isco Combiflash, 120 g. Redisep Gold, Ethyl Acetate, Heptane gradient, 0-100% ethyl acetate over 15 minutes) to afford 10 mg of the desired tricyclic indole product.

The product fractions were then combined and concentrated in vacuo (SpeedVac) and the resulting oils were used in screening.

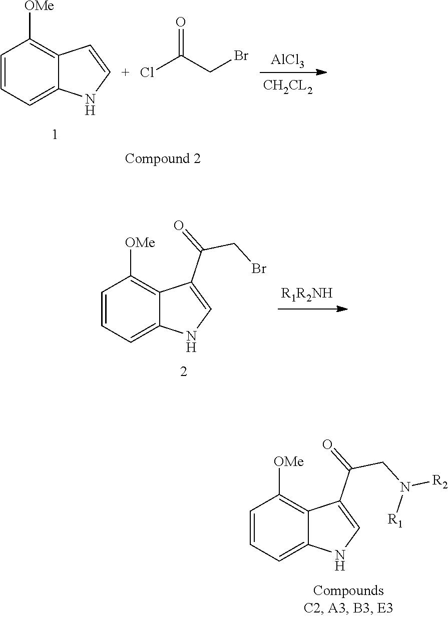

### Compound 2

1.57 grams of bromoacetylchloride was charged to an addition funnel along with 5 mL of dichloromethane and outfitted to a 50 mL 3-neck round bottom flask containing 1.47 g of 4-Methoxyindol (1) dissolved in 15 mL of dichloromethane. 75 mg of aluminum trichloride as a solid was added and the reaction mixture was purged with nitrogen, and stirred at room temperature for 30 minutes. The bromoacetyl chloride solution was then added dropwise over 30 minutes to the stirred reaction mixture, and the reaction mixture stirred for 15 hours at room temperature. The reaction mixture was then assayed for completion and 15 mL of 0.1 M sodium potassium tartrate was added via syringe and the mixture stirred for an additional 5 minutes before transferring to a separatory funnel.

The organic layer was then extracted with 15 mL of saturated brine solution collected and dried over sodium sulfate. Solvent was removed by rotary evaporator, and the solution purified by silica-gel chromatography (Isco Combiflash, 120 g. Redisep Gold, Ethyl Acetate, Heptane gradient, 0-100% ethyl acetate over 15 minutes). The main product fractions were collected and concentrated by rotary evaporator, at which time the product precipitated after removal of the ethyl acetate. The product was then filtered off, washed with 10 mL of Heptane, and dried under vacuum before use, to afford 1.95 grams of compound 2 (73%).

### Compounds C2, A3, B3 and E3

In a 1 dram vial outfitted with a stir bar, a solution of 50 mg of 2, dissolved in 500 μL of n-methyl pyrollidone, was combined with 4 equivalents of amine charged all in one portion. The reaction mixtures were heated to 60 C in an aluminum heating block with stirring, and determined to be complete after 3 hours as assayed by RP-C18 UPLC.

2 ml of water was added to quench the reactions and the mixture transferred to a separatory funnel where 10 mL of ethyl acetate, 3 mL of 0.1 M NaOH was added and the organic layer was extracted, followed by extraction with 5 mL of saturated brine solution. The organic layer was then dried over sodium sulfate, the mixture concentrated by rotary evaporation, where it was purified by silica gel flash chromatography. (Isco Combiflash, 12 g, Redisep Gold, Ethyl Acetate, Heptane gradient, 0-100% ethyl acetate over 15 minutes).

The product fractions were then combined and concentrated in vacuo (SpeedVac), and the resulting oils were used in screening.

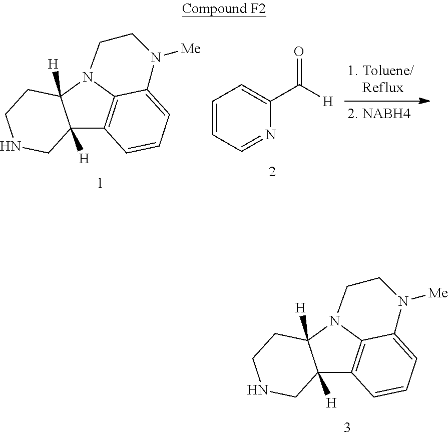

### Compound F2

23 mg of starting product (above referenced as “1”) was charged to a 14 mL open test tube along with 3 mL of Toluene, 15 mg of 2 (shown above), and 20 μL of acetic acid. The reaction mixture was heated to reflux temperature for 30 minutes. After 30 minutes the reaction mixture was allowed to cool, then 25 mg of sodium borohydride was added as a solid and the mixture stirred for 1 hour. 5 ml of water was added, and the reaction mixture extracted. The top layer was collected and dried with sodium sulfate, followed by concentration in vacuo to a brown oil which was purified by silica gel chromatography, (Isco Combiflash, 12 g, Redisep Gold, ethyl acetate, heptane gradient, 0-100% ethyl acetate over 15 minutes) to afford 18 mg of the desired amine product, 3 (shown above).

The product fractions were then combined and concentrated in vacuo (SpeedVac), and the resulting oils were used in screening.

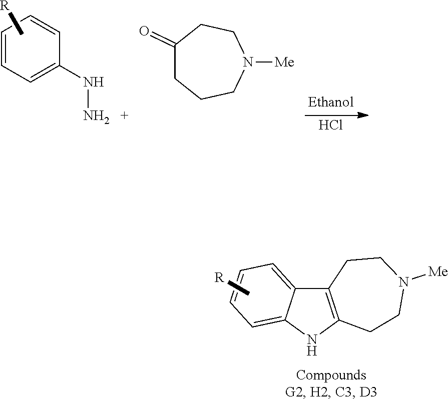

### Compounds G2, H2, C3, D3

Embodiments also include methods of synthesis of compounds G2, H2, C3 and D3. The compounds were prepared by methods known in the art (see, e.g., WO2021/176599A1). Briefly, 0.5 mMole of Aryl Hydrazine, and 0.5 mMole of Ketone 4 mL of Ethanol and 6 eq of 37% HCl were charged to an 8 mL sealed vial. The reaction mixture was heated to 100° C. overnight. The next day solvent was removed by rotary evaporator, and compounds taken up in 10 mL dichloromethane. and extracted with 10 mL of 0.1 M HCl, two times. The dichloromethane layer was dried with sodium sulfate and concentrated to an oil.

The oil was purified by silica gel flash chromatography. (Isco Combiflash, 12 g, Redisep Gold, Methanol, Dichloromethane (both containing 2% Ammonium Hydroxide) gradient, 0-50% methanol over 15 minutes). The product fractions were then combined and concentrated in vacuo (SpeedVac), and the resulting oils were used in screening.

### Analogs

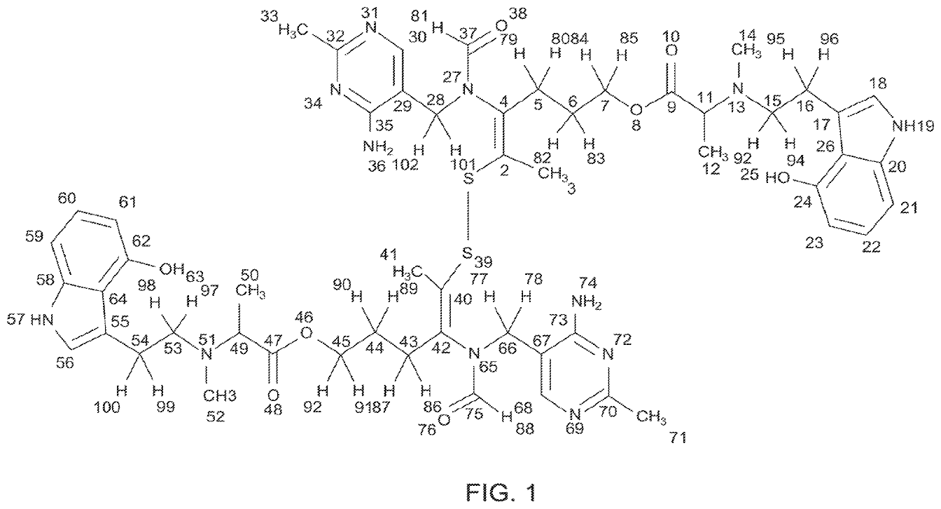

Embodiments also include analogs of Somahol. FIG. 1 depicts the structure of Somahol with numbered atoms. The molecule can include five or more additional methyl groups between carbon 6 and 7 as well as 43 and 44. An oxygen at portion 10 and 48 can be replaced by hydroxyl groups. Sulfur can be replaced by phosphorous. Alternatively, sulfur can be replaced by tri-phosphate backbone attached to a fatty acid chain or sulfur can be replaced by a lipoprotein in various forms such as cholesterol impregnated with magnesium. Further, the carbon (54 and 55) covalent bond can be replaced by an ionic bond using salt components at position 54 such as potassium and phosphate or other iterations of salts. A functional group beyond 54 can be directly attached to the phosphate center in another iteration.

Embodiments also include pro-drugs of therapeutic molecule disclosed herein.

In one embodiment, one or more therapeutic molecules described herein can stimulate synaptic neuroplasticity. Synaptic neuroplasticity can be defined as the property of synapses to strengthen or weaken in response to changes in presynaptic activity. This plasticity often manifests as a change to the number of synapses on a particular neuron, thereby altering brain-wide neural connectivity on a long-term scale. Based on molecular mechanisms obtained from biopsied brain tissue, Applicants have identified mechanisms for remodeling the addicted brain and resolving addiction. Applicants propose that the compound of Formula I (i.e., Somahol) can have beneficial effects on the brain which may promote limbic restructuring.

Without being bound by theory, Applicants propose that the molecule affects the upstream regulators of FOS, JUN, BDNF, CDC42 and CCL2 via cohort activity of upstream interaction. These direct upstream interactions are a cohort of all 5-HT receptor agonism via direct binding, Ga12 agonism, allosteric regulation of acetyl-CoA metabolism including regulatory acetylation of proteins and acetylcholine biosynthesis, increased MDH2 coding via promotion of acetylation and agonism of THDP pathways via coenzyme mimicry.

Based on Applicants' mechanistic model, the downstream effects of these interactions result in homeostasis of the stimulation of FosB, JUN, BDNF and decrease relative abundance of CCL2 via combined activation of 5-HT7R, 5-HT2AR, oxytocin, G couple protein, mitochondrial stimulation and downstream feedback. Activities in this downstream chain include promotion of oxytocin, acetylcholine and dopamine.

Accordingly, in one embodiment, Somahol (or other therapeutic molecule) can modulate one or more G protein-coupled receptor and ligand-gated ion channels found in the central and peripheral nervous systems such as the 5-HT receptor. In embodiments, Somahol (or other therapeutic molecule) affects one or more proto-oncogenes such as FOS, JUN, BDNF, CDC42 or CCL2. In one embodiment, Somahol modulates levels of oxytocin, acetylcholine and/or dopamine.

These combined activities are involved in the increase of neuroplasticity. For example, the therapeutic molecules described herein can exhibit mechanisms through both allosteric and direct binding which effectively improve dendrite formation via Ga12 signaling including cdc42 stimulation. The molecules can exhibit effects on enhanced neurohormone signaling including the aforementioned effects on oxytocin, acetylcholine and dopamine most notably in the limbic cortex, cingulate gyrus, raphe nuclei, parahippocampal gyrus, dentate gyrus, hippocampus, subicular complex, amygdala, septal area and hypothalamus.

### Pathways

An essential characteristic of the nervous system is its capacity to reshape functional connectivity in response to physiological and environmental cues. Endogenous signals, including neuropeptides, govern nervous system plasticity. Particularly, oxytocin has been recognized for its role in mediating activity-dependent circuit changes. These oxytocin-dependent changes occur at the synaptic level and consequently shape the cellular composition of circuits.

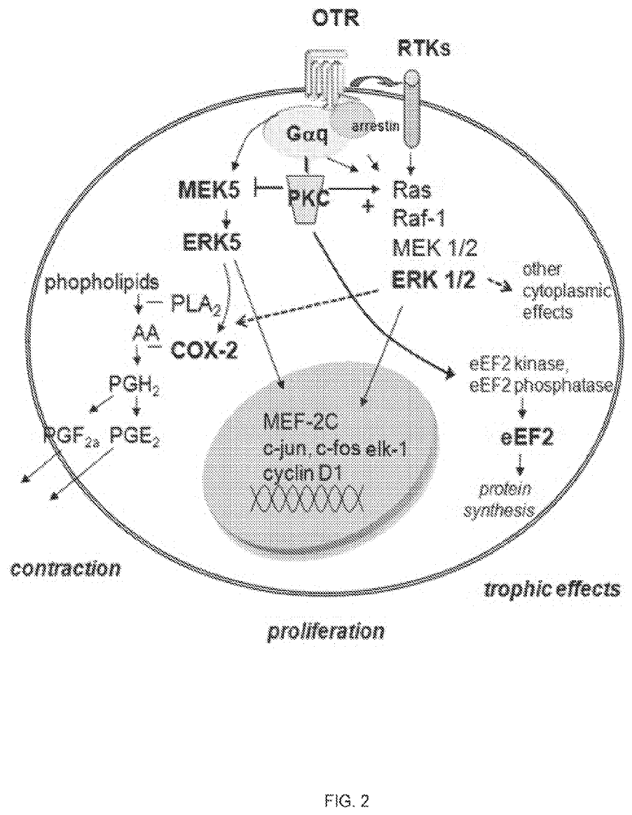

FIG. 2 depicts the oxytocin signaling pathway. Alterations in oxytocin signaling can disturb neuronal maturation and may have short-term and long-term pathological consequences. Aberrant regulation of oxytocin signaling is associated with the etiology of neurodevelopmental disorders. Recent studies have demonstrated synaptic dysfunctions in neurodevelopmental disorders. Accordingly, pathogenic mechanisms can be targets of therapeutic intervention.

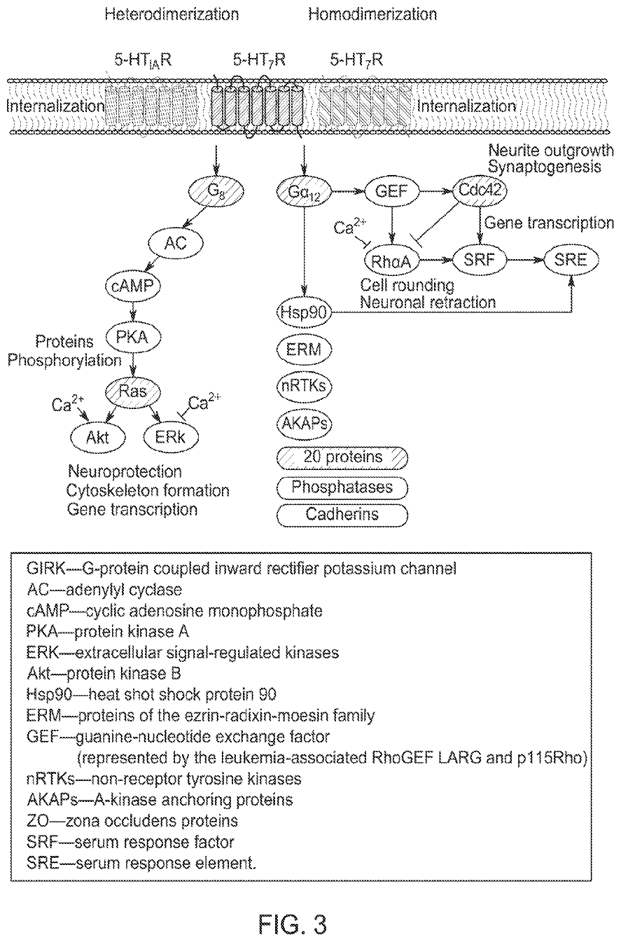

The neurotransmitter serotonin (5-HT) plays a major a role in behavioral and psychophysiological functions such as behavioral inhibition, appetite regulation, mood, cognitive functions, thermoregulation and addictive behaviors. The serotonin (5-HT) neurotransmitter system is of particular interest and multiple 5-HT receptors are thought to play significant roles in alcohol and drug self-administration and the development of drug dependence. A serotonin receptor agonist is an agonist of one or more serotonin receptors. They activate serotonin receptors in a manner similar to that of serotonin (5-hydroxytryptamine; 5-HT), a neurotransmitter and hormone and the endogenous ligand of the serotonin receptors. FIG. 3 depicts the 5-HT receptors and related actions. Somahol selectively activates the 5-HT7 receptor (i.e., 5-HT-R) which ultimately activates dendritic outreach.

Accordingly, in embodiments, Somahol (or other therapeutic molecule) can modulate one or more of behavioral inhibition, appetite regulation, mood, cognitive functions, thermoregulation and addictive behavior. In other embodiments, Somahol can be administered to treat addiction, alcohol dependency and/or prevent the development of drug dependence.

Somahol was studied for its agonist abilities. The lowest Ki values were observed with the 5-HT2B and 5-HT7 receptors. Varied 5-HT receptor agonism (as shown in Table 1 below) is coupled with other portions of the molecule which exhibit high binding affinity at DJ-1 (PARK7) and amyloid beta (a4). The molecule may also promote the relative abundance of proteins, MARCS, VATA, VATB, THY1 and HPLN1 through mechanisms currently being investigated.

TABLE 1

| Receptor | Kᵢ (nM) |
|---|---|
| 5-HT_{1A} | 50 |
| 5-HT_{1B} | 220 |
| 5-HT_{1D} | 38 |
| 5-HT_{1E} | 55 |
| 5-HT_{2A} | 150 |
| 5-HT_{2B} | 7 |
| 5-HT_{2C} | 99 |
| 5-HT₅ | 88 |
| 5-HT₆ | 60 |
| 5-HT₇ | 5 |

The 5-HT₇ receptor is involved in thermoregulation, circadian rhythm, learning and memory, and sleep. It is also speculated that this receptor may be involved in mood regulation, suggesting that it may be a useful target in the treatment of depression. FIG. 2 is a schematic representation of signaling pathways regulated by the 5-HT₇ receptor. The left section lists effects mediated by Gs-proteins. Similarly, the right section lists G12-mediated signaling processes.

In embodiments, Somahol can be administered to a patient as a 5-HT₇ receptor agonist. In embodiments, Somahol can modulate circadian rhythm, learning and memory, sleep, mood regulation and/or depression.

In embodiments, a therapeutic molecule disclosed herein can be administered to a patient as a 5-HT₇ receptor agonist. In embodiments, the molecule can modulate circadian rhythm, learning and memory, sleep, mood regulation and/or depression. The molecule can be A1, B1, C1, D1, E1, F1, G1, H1, A2, B2, C2, D2, E2, F2, G2, H2, A3, B3, C3, D3 and/or E3.

### RNASE1

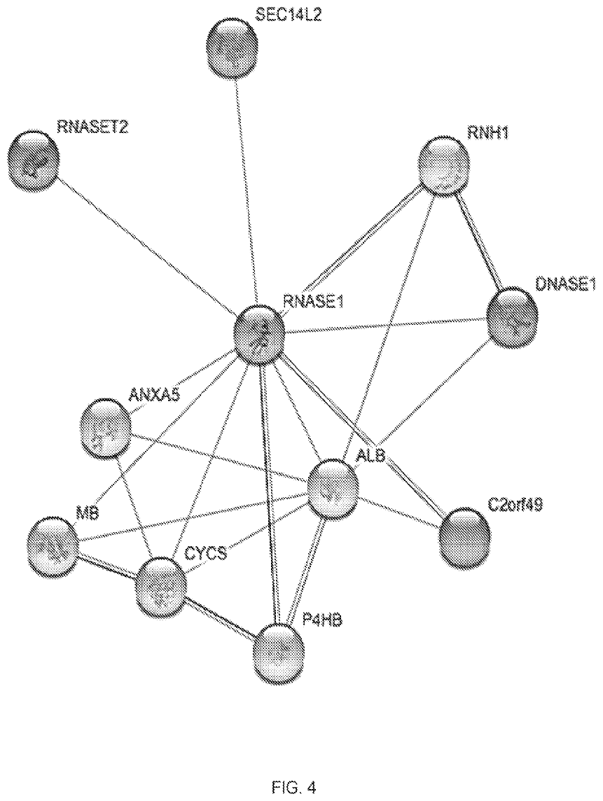

Ribonuclease pancreatic is an enzyme that in humans is encoded by the RNASE1 gene. The gene encodes a member of the pancreatic type of secretory ribonucleases, a subset of the ribonuclease A super-family. The encoded endonuclease cleaves internal phosphodiester RNA bonds on 3′-side of pyrimidine bases. FIG. 4 depicts RNASE I interactions with adjacent genes.

Functional partners of RNASE1 include:
1. RNH1: Ribonuclease inhibitor; Ribonuclease inhibitor which inhibits RNASE1, RNASE2, and ANG. This gene may play a role in redox homeostasis.
2. SE14L2: SEC14-like protein 2; Carrier protein. Binds to some hydrophobic molecules and promotes their transfer between the different cellular sites. Binds with high affinity to alpha-tocopherol. Also binds with a weaker affinity to other tocopherols and to tocotrienols. May have a transcriptional activator activity via its association with alpha-tocopherol. Probably recognizes and binds some squalene structure, suggesting that it may regulate cholesterol biosynthesis by increasing the transfer of squalene to a metabolic active pool in the cell.
3. ANXA5 (Annexin A5) This protein is an anticoagulant protein that acts as an indirect inhibitor of the thromboplastin-specific complex, which is involved in the blood coagulation cascade.
4. RNASET2 (Ribonuclease T2) This protein has ribonuclease activity, with higher activity at acidic pH. It is likely involved in lysosomal degradation of ribosomal RNA (by similarity) and may play a role in cellular RNA catabolism
5. MB (Myoglobin) MB serves as a reserve supply of oxygen and facilitates the movement of oxygen within muscles.
6. C2orf49 (Ashwn; Chromosome 2 open reading frame 49).
7. ALB (Serum albumin) It is the main protein of plasma, has a good binding capacity for water, Ca (2+), Na (+), K (+), fatty acids, hormones, bilirubin and drugs. Its main function is the regulation of the colloidal osmotic pressure of blood. Major zinc transporter in plasma, typically binds about 80% of all plasma zinc.
8. DNASE1 (Deoxyribonuclease-1) Among other functions, DNASE1 seems to be involved in cell death by apoptosis. It binds specifically to G-actin and blocks actin polymerization.
9. CYCS (Cytochrome c; Electron carrier protein) The oxidized form of the cytochrome c heme group can accept an electron from the heme group of the cytochrome c1 subunit of cytochrome reductase. Cytochrome c then transfers this electron to the cytochrome oxidase complex, the final protein carrier in the mitochondrial electron-transport chain.
10. P4HB (Protein disulfide-isomerase) This multifunctional protein catalyzes the formation, breakage and rearrangement of disulfide bonds. At the cell surface, it seems to act as a reductase that cleaves disulfide bonds of proteins attached to the cell. Therefore, it may cause structural modifications of exofacial proteins. Inside the cell, it appears to form/rearrange disulfide bonds of nascent proteins. At high concentrations, it functions as a chaperone that inhibits aggregation of misfolded proteins. At low concentrations, it facilitates aggregation (anti-chaperone activity).

Accordingly, in embodiments, Somahol (or other therapeutic molecule disclosed herein) can be administered to modulate RNASE1 levels and/or activity. In embodiments, functional partners of RNASE1 include one or more of RNH1, SE14L2, ANXA5, RNASET2, MB, C2orf49, ALB, DNASE1, CYCS and P4HB. FIG. 1 depicts RNASE1 interacting with adjacent genes or “functional partners.”

### CCL2

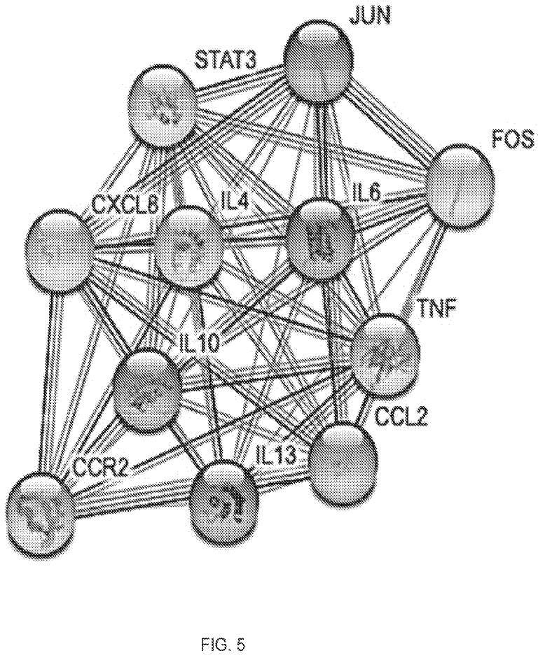

The chemokine (C-C motif) ligand 2 (CCL2) can also be referred to as monocyte chemoattractant protein 1 (MCP1) and small inducible cytokine A2. CCL2 is a small cytokine that belongs to the CC chemokine family. It attracts monocytes and basophils but not neutrophils or eosinophils. It also augments monocyte anti-tumor activity and has been implicated in the pathogenesis of diseases characterized by monocytic infiltrates, like psoriasis, rheumatoid arthritis or atherosclerosis. Recent studies have demonstrated that CCL2-treated neural stem cells showed significantly increased capacity for self-renewal, proliferation and neuronal differentiation. Similarly, FIG. 5 depicts CCL2 interactions with adjacent genes.

Functional Partners of CCL2 include:
1. CCR2 (C-C chemokine receptor type 2; Receptor for the CCL2, CCL7 and CCL13 chemokines). CCR2 is a receptor for the beta-defensin DEFB106A/DEFB106B. It transduces a signal by increasing intracellular calcium ion levels (by similarity). Upon CCL2 ligation, it mediates chemotaxis and migration induction through the activation of the PI3K cascade, the small G protein Rac and lamellipodium protrusion.
2. IL10 (Interleukin-10) IL10 inhibits the synthesis of a number of cytokines, including IFN-gamma, IL-2, IL-3, TNF and GM-CSF produced by activated macrophages and by helper T-cells.
3. IL4 (Interleukin-4) IL4 participates in at least several B-cell activation processes as well as of other cell types. It is a co-stimulator of DNA-synthesis. It induces the expression of class II MHC molecules on resting B-cells. It enhances both secretion and cell surface expression of IgE and IgG1. It also regulates the expression of the low affinity Fc receptor for IgE (CD23) on both lymphocytes and monocytes. It also positively regulates IL31RA expression in macrophages.
4. IL6 (Interleukin-6) IL6 is a cytokine with a wide variety of biological functions. It is a potent inducer of the acute phase response. It plays an essential role in the final differentiation of B-cells into Ig-secreting cells Involved in lymphocyte and monocyte differentiation. It acts on B-cells, T-cells, hepatocytes, hematopoietic progenitor cells and cells of the CNS. Required for the generation of T (H) 17 cells. It also acts as a myokine and is discharged into the bloodstream after muscle contraction and acts to increase the breakdown of fats and to improve insulin resistance.
5. STAT3 (Signal transducer and activator of transcription 3) STAT3 is a signal transducer and transcription activator that mediates cellular responses to interleukins, KITLG/SCF, LEP and other growth factors. Once activated, it recruits coactivators, such as NCOA1 or MED1, to the promoter region of the target gene. May mediate cellular responses to activated FGFR1, FGFR2, FGFR3 and FGFR4. Binds to the interleukin-6 (IL-6)-responsive elements identified in the promoters of various acute-phase protein genes. It is activated by IL31 through IL31RA.
6. IL13 (Interleukin-13; Cytokine) IL3 inhibits inflammatory cytokine production. It synergizes with IL2 in regulating interferon-gamma synthesis and may be critical in regulating inflammatory and immune responses. Positively regulates IL31RA expression in macrophages.
7. CXCL8 (Interleukin-8) IL-8 is a chemotactic factor that attracts neutrophils, basophils, and T-cells, but not monocytes. It is also involved in neutrophil activation. It is released from several cell types in response to an inflammatory stimulus. IL-8(6-77) has a 5-10-fold higher activity on neutrophil activation, IL-8(5-77) has increased activity on neutrophil activation and IL-8(7-77) has a higher affinity to receptors CXCR1 and CXCR2 as compared to IL-8(1-77), respectively.
8. JUN (Transcription factor AP-1) JUN is a transcription factor that recognizes and binds to the enhancer heptamer motif 5′-TGA[CG]TCA-3′. It promotes activity of NR5A1 when phosphorylated by HIPK3 leading to increased steroidogenic gene expression upon CAMP signaling pathway stimulation and is involved in activated KRAS-mediated transcriptional activation of USP28 in colorectal cancer (CRC) cells. It binds to the USP28 promoter in colorectal cancer (CRC) cells and is a basic leucine zipper protein.
9. TNF This gene encodes a multifunctional proinflammatory cytokine that belongs to the tumor necrosis factor (TNF) superfamily. This cytokine is mainly secreted by macrophages. It can bind to, and thus functions through its receptors TNFRSF1A/TNFR1 and TNFRSF1B/TNFBR. This cytokine is involved in the regulation of a wide spectrum of biological processes including cell proliferation, differentiation, apoptosis, lipid metabolism, and coagulation. This cytokine has been implicated in a variety of diseases, including autoimmune diseases, insulin resistance, psoriasis, rheumatoid arthritis, ankylosing spondylitis, tuberculosis, autosomal dominant polycystic kidney disease, and cancer. Mutations in this gene affect susceptibility to cerebral malaria, septic shock, and Alzheimer disease. Knockout studies in mice also suggested the neuroprotective function of this cytokine.
10. FOS The Fos gene family consists of 4 members: FOS, FOSB, FOSL1, and FOSL2. These genes encode leucine zipper proteins that can dimerize with proteins of the JUN family, thereby forming the transcription factor complex AP-1. As such, the FOS proteins have been implicated as regulators of cell proliferation, differentiation, and transformation. In some cases, expression of the FOS gene has also been associated with apoptotic cell death.

Accordingly, in embodiments, Somahol (or other therapeutic molecule disclosed herein) can be administered to modulate CCL2 levels and/or activity. In embodiments, functional partners of CCL2 include one or more of CR2, IL10, IL4, IL6, STAT3, IL13, CXCL8, JUN, TNF and FOS.

### FOSB

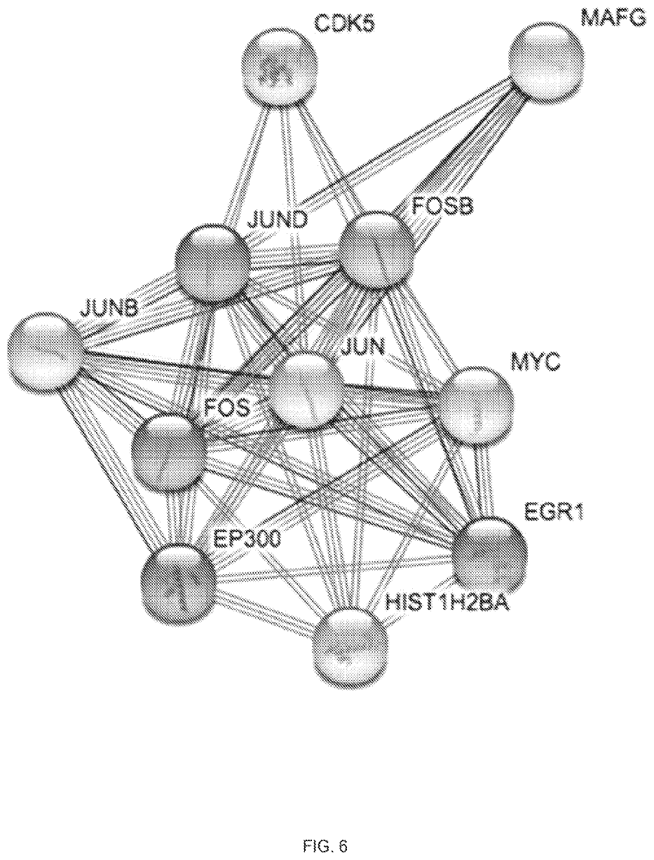

Protein fosB, also known as FOS, FosB and G0/G1 switch regulatory protein 3 (G0S3), is a protein that in humans is encoded by the FBJ murine osteosarcoma viral oncogene homolog B (FOSB) gene. FIG. 5 depicts FOS interactions with adjacent genes. FIG. 6 depicts FosB interactions with adjacent genes.

Functional Partners of FOSB include:
1. JUN This gene is the putative transforming gene of avian sarcoma virus 17. It encodes a protein which is highly similar to the viral protein, and which interacts directly with specific target DNA sequences to regulate gene expression. This gene is intronless and is mapped to 1p32-p31, a chromosomal region involved in both translocations and deletions in human malignancies.
2. JUND The protein encoded by this intronless gene is a member of the JUN family, and a functional component of the AP1 transcription factor complex. This protein has been proposed to protect cells from p53-dependent senescence and apoptosis. Alternative translation initiation site usage results in the production of different isoforms.
3. JUNB Transcription factor jun-B; Transcription factor involved in regulating gene activity following the primary growth factor response. Binds to the DNA sequence 5′-TGA[CG]TCA-3′; Belongs to the bZIP family.
4. FOS The Fos gene family consists of 4 members: FOS, FOSB, FOSL1, and FOSL2. These genes encode leucine zipper proteins that can dimerize with proteins of the JUN family, thereby forming the transcription factor complex AP-1. As such, the FOS proteins have been implicated as regulators of cell proliferation, differentiation, and transformation. In some cases, expression of the FOS gene has also been associated with apoptotic cell death.
5. CDK5 This gene encodes a proline-directed serine/threonine kinase that is a member of the cyclin-dependent kinase family of proteins. Unlike other members of the family, the protein encoded by this gene does not directly control cell cycle regulation. Instead, the protein, which is predominantly expressed at high levels in mammalian postmitotic central nervous system neurons, functions in diverse processes such as synaptic plasticity and neuronal migration through phosphorylation of proteins required for cytoskeletal organization, endocytosis and exocytosis, and apoptosis. In humans, an allelic variant of the gene that results in undetectable levels of the protein has been associated with lethal autosomal recessive lissencephaly-7. Alternative splicing results in multiple transcript variants.
6. EP300 This gene encodes the adenovirus E1A-associated cellular p300 transcriptional co-activator protein. It functions as histone acetyltransferase that regulates transcription via chromatin remodeling and is important in the processes of cell proliferation and differentiation. It mediates cAMP-gene regulation by binding specifically to phosphorylated CREB protein. This gene has also been identified as a co-activator of HIF1A (hypoxia-inducible factor 1 alpha), and thus plays a role in the stimulation of hypoxia-induced genes such as VEGF. Defects in this gene are a cause of Rubinstein-Taybi syndrome and may also play a role in epithelial cancer.
7. MAFG Globin gene expression is regulated through nuclear factor erythroid-2 (NFE2) elements located in enhancer-like locus control regions positioned many kb upstream of alpha- and beta-gene clusters (summarized by Blank et al., 1997 [PubMed 9166829]). NFE2 DNA-binding activity consists of a heterodimer containing a ubiquitous small Maf protein (MafF, MIM 604877; MafG; or MafK, MIM 600197) and the tissue-restricted protein p45 NFE2 (MIM 601490). Both subunits are members of the activator protein-1-like superfamily of basic leucine zipper (bZIP) proteins.
8. EGR1 The protein encoded by this gene belongs to the EGR family of C2H2-type zinc-finger proteins. It is a nuclear protein and functions as a transcriptional regulator. The products of target genes it activates are required for differentiation and mitogenesis. Studies suggest this is a cancer suppressor gene.
9. MYC (Myc proto-oncogene protein) MYC is a transcription factor that binds DNA in a non-specific manner, yet also specifically recognizes the core sequence 5′-CAC[GA]TG-3′. Activates the transcription of growth-related genes. Binds to the VEGFA promoter, promoting VEGFA production and subsequent sprouting angiogenesis; Basic helix-loop-helix proteins.
10. HIST1H2BA Histones are basic nuclear proteins that are responsible for the nucleosome structure of the chromosomal fiber in eukaryotes. Nucleosomes consist of approximately 146 bp of DNA wrapped around a histone octamer composed of pairs of each of the four core histones (H2A, H2B, H3, and H4). The chromatin fiber is further compacted through the interaction of a linker histone, H1, with the DNA between the nucleosomes to form higher order chromatin structures. This gene is intronless and encodes a replication-dependent histone that is a testis/sperm-specific member of the histone H2B family. Transcripts from this gene contain a palindromic termination element.

Accordingly, in embodiments, Somahol (or other therapeutic molecule disclosed herein) can be administered to modulate FosB levels and/or activity. In embodiments, functional partners of FosB include one or more of JUN, JUND, JUNB, FOS (i.e., another member), CDK5, EP300, MAFG, EGR1, MYC and HIST1H2BA.

### SLC39A8

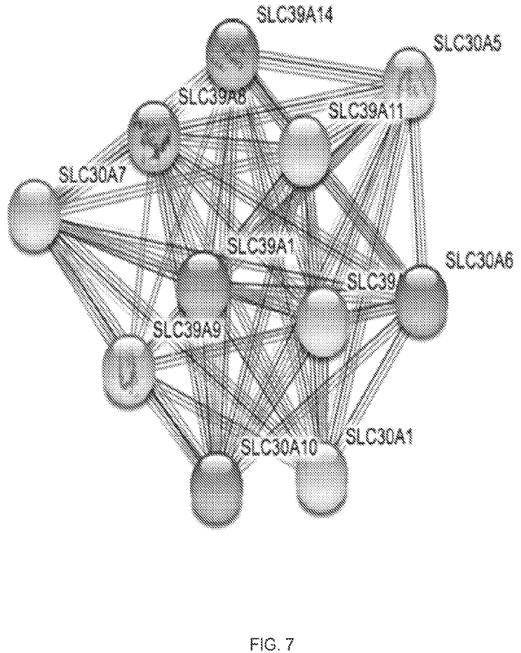

The SLC39A8 gene encodes a member of the SLC39 family of solute-carrier genes, which show structural characteristics of zinc transporters. The encoded protein is glycosylated and found in the plasma membrane and mitochondria, and functions in the cellular import of zinc at the onset of inflammation. It is also thought to be the primary transporter of the toxic cation cadmium, which is found in cigarette smoke. Multiple transcript variants encoding different isoforms have been found for this gene. Additional alternatively spliced transcript variants of this gene have been described, but their full-length nature is not known. FIG. 7 depicts SLC39A8 interacting with adjacent genes.

Functional Partners of SLC39A8 include:
1. SLC30A1 (Zinc transporter 1) SLC30A1 may be involved in zinc transport out of the cell; Belongs to the cation diffusion facilitator (CDF) transporter (TC 2.A.4).
2. SLC39A14 (Zinc transporter ZIP14) SLC39A14 is a broad-scope metal ion transporter with a preference for zinc uptake. It also mediates cellular uptake of non transferrin-bound iron; Belongs to the ZIP transporter (TC 2.A.5) family.
3. SLC39A2 This gene encodes a member of the ZIP family of metal ion transporters. The encoded protein functions as a zinc transporter. Mutations in this gene may be associated with susceptibility to carotid artery disease. Multiple transcript variants have been described.
4. SLC39A1 This gene encodes a member of the zinc-iron permease family. The encoded protein is localized to the cell membrane and acts as a zinc uptake transporter. This gene has been linked to prostate cancer, breast cancer, and Alzheimer's disease. Alternative splicing results in multiple transcript variants.
5. SLC39A9 (Zinc transporter ZIP9) This gene may act as a zinc-influx transporter; Belongs to the ZIP transporter (TC 2.A.5) family.
6. SLC30A10 This gene is highly expressed in the liver and is inducible by manganese. Its protein product appears to be critical in maintaining manganese levels and has higher specificity for manganese than zinc. Loss of function mutations appear to result in a pleomorphic phenotype, including dystonia and adult-onset parkinsonism. Alternatively spliced transcript variants have been observed for this gene.
7. SLC30A7 Zinc functions as a cofactor for numerous enzymes, nuclear factors, and hormones and as an intra- and intercellular signal ion. Members of the zinc transporter (ZNT)/SLC30 subfamily of the cation diffusion facilitator family, such as SLC30A7, permit cellular efflux of zinc.
8. SLC30A6 This gene encodes a member of a family of proteins that function as zinc transporters. This protein can regulate subcellular levels of zinc in the Golgi and vesicles. Expression of this gene is altered in the Alzheimer's disease brain plaques.
9. SLC30A5 This gene encodes a member of the SLC30A/ZnT family of zinc transporter proteins. ZnT proteins mediate both cellular zinc efflux and zinc sequestration into membrane-bound organelles. The encoded protein plays a role in the early secretory pathway as a heterodimer with zinc transporter 6, and may also regulate zinc sequestration into secretory granules of pancreatic beta cells. Alternatively spliced transcript variants encoding multiple isoforms have been observed for this gene, and a pseudogene of this gene is located on the long arm of chromosome 19.
10. SLC39A11 Zinc transporter ZIP11; Functions as a cellular zinc transporter.

Accordingly, in embodiments, Somahol (or other therapeutic molecule disclosed herein) can be administered to modulate SLC39A8 levels and/or activity. In embodiments, functional partners of SLC39A8 include one or more of SLC30A1, SLC39A14, SLC39A2, SLC39A1, SLC39A9, SLC30A10, SLC30A7, SLC30A6, SLC30A5 and SLC39A11.

### C9orf135

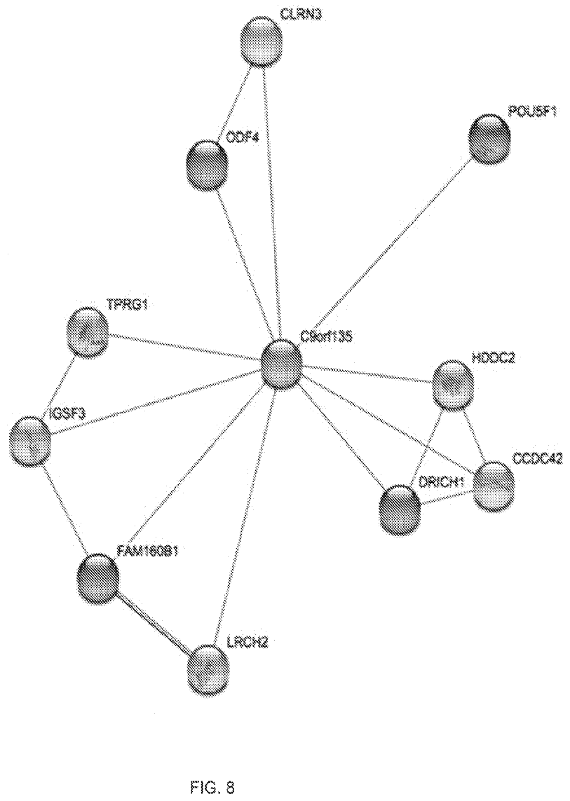

C9orf135 is a gene that encodes a 229-amino-acid protein. It is located on Chromosome 9 of the Homo sapiens genome at 9q12.21. The protein has a transmembrane domain from amino acids 124-140 and a glycosylation site at amino acid 75. C9orf135 has been associated with neural development. FIG. 8 depicts C9ORF135 interacting with adjacent genes.

Functional Partners of C9orf135 include:
1. TPRG1 (Tumor Protein P63 Regulated 1) TPRG1 is a Protein Coding gene. Diseases associated with TPRG1 include Mixed Liposarcoma. An important paralog of this gene is TPRG1L.
2. DRICH1 (Aspartate Rich 1) DRICH1 is a Protein Coding gene. Diseases associated with DRICH1 include Childhood Acute Lymphocytic Leukemia and Retinitis Pigmentosa 49. An important paralog of this gene is C22orf42.
3. CCDC42 (Coiled-coil domain-containing protein 42) CCDC42 is required for sperm development.
4. ODF4 This gene encodes a protein that is localized in the outer dense fibers of the tails of mature sperm. This protein is thought to have some important role in the sperm tail. Alternative splicing results in multiple transcript variants.
5. HDDC2 (HD domain containing 2) Belongs to the HDDC2 family.
6. FAM160B1 (FHIP2A, FHF Complex Subunit HOOK Interacting Protein 2A) Is a Protein Coding gene. Diseases associated with FHIP2A include Syndromic Intellectual Disability and Alacrima, Achalasia, And Mental Retardation Syndrome. An important paralog of this gene is FHIP2B.
7. IGSF3 The protein encoded by this gene is an immunoglobulin-like membrane protein containing several V-type Ig-like domains. A mutation in this gene has been associated with bilateral nasolacrimal duct obstruction (LCDD).
8. POU51 This gene encodes a transcription factor containing a POU homeodomain that plays a key role in embryonic development and stem cell pluripotency. Aberrant expression of this gene in adult tissues is associated with tumorigenesis. This gene can participate in a translocation with the Ewing's sarcoma gene on chromosome 21, which also leads to tumor formation. Alternative splicing, as well as usage of alternative AUG and non-AUG translation initiation codons, results in multiple isoforms. One of the AUG start codons is polymorphic in human populations. Related pseudogenes have been identified on chromosomes 1, 3, 8, 10, and 12.
9. LRCH2 This gene encodes a member of the leucine-rich repeat and calponin homology domain-containing protein family. These family members contain multiple N-terminal leucine-rich repeats, in addition to a C-terminal calponin homology domain, a type of domain that mediates interactions with actin filaments. These proteins are conserved across animal species, and studies of a similar Drosophila protein indicate a function as a cytoskeletal scaffolding protein. Alternative splicing of this gene results in multiple transcript variants.
10. CLRN3 (Clarin 3) CLRN3 is a Protein Coding gene. Diseases associated with CLRN3 include Usher Syndrome and Gastric Tubular Adenocarcinoma. An important paralog of this gene is CLRN2.

Accordingly, in embodiments, Somahol (or other therapeutic molecule disclosed herein) can be administered to modulate C9orf135 levels and/or activity. In embodiments, functional partners of C9orf135 include one or more of TPRG1, DRICH1, CCDC42, ODF4, HDDC2, FAM160B1, IGSF3, POU51, LRCH2 and CLRN3.

### ENTPD4

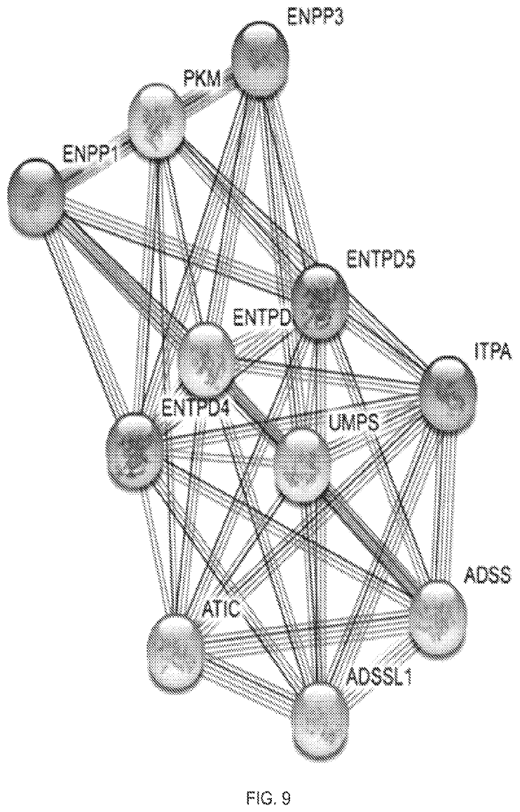

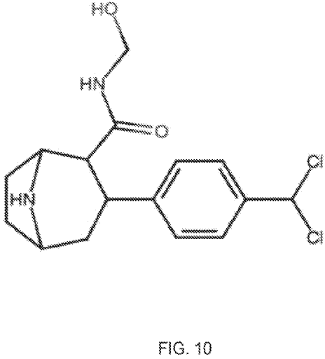

The ENTPD4 gene encodes a member of the apyrase protein family. Apyrases are enzymes that catalyze the hydrolysis of nucleotide diphosphates and triphosphates in a calcium or magnesium-dependent manner. The encoded protein is an endo-apyrase and may play a role in salvaging nucleotides from lysosomes. Alternatively spliced transcript variants encoding multiple isoforms have been observed for this gene, and these isoforms may differ in divalent cation dependence and substrate specificity. FIG. 9 depicts ENTPD4 interacting with adjacent genes.

Functional Partners of ENTPD4 include:
1. ADSSL1 This gene encodes a member of the adenylosuccinate synthase family of proteins. The encoded muscle-specific enzyme plays a role in the purine nucleotide cycle by catalyzing the first step in the conversion of inosine monophosphate (IMP) to adenosine monophosphate (AMP). Mutations in this gene may cause adolescent onset distal myopathy. Alternative splicing results in multiple transcript variants.
2. ITPA (Inosine triphosphate pyrophosphatase) ITPA is a pyrophosphatase that hydrolyzes the non-canonical purine nucleotides inosine triphosphate (ITP), deoxyinosine triphosphate (dITP) as well as 2′-deoxy-N-6-hydroxylaminopurine triposphate (dHAPTP) and xanthosine 5′-triphosphate (XTP) to their respective monophosphate derivatives. The enzyme does not distinguish between the deoxy- and ribose forms. Probably excludes non-canonical purines from RNA and DNA precursor pools, thus preventing their incorporation into RNA and DNA and avoiding chromosomal lesions.
3. ATIC This gene encodes a bifunctional protein that catalyzes the last two steps of the de novo purine biosynthetic pathway. The N-terminal domain has phosphoribosylaminoimidazolecarboxamide formyltransferase activity, and the C-terminal domain has IMP cyclohydrolase activity. A mutation in this gene results in AICA-ribosiduria.
4. ENPP3 The protein encoded by this gene belongs to a series of ectoenzymes that are involved in hydrolysis of extracellular nucleotides. These ectoenzymes possess ATPase and ATP pyrophosphatase activities and are type II transmembrane proteins. Expression of the related rat mRNA has been found in a subset of immature glial cells and in the alimentary tract. The corresponding rat protein has been detected in the pancreas, small intestine, colon, and liver. The human mRNA is expressed in glioma cells, prostate, and uterus. Expression of the human protein has been detected in uterus, basophils, and mast cells. Two transcript variants, one protein coding and the other non-protein coding, have been found for this gene.
5. ADSS This gene encodes the enzyme adenylosuccinate synthetase which catalyzes the first committed step in the conversion of inosine monophosphate to adenosine monophosphate. A pseudogene of this gene is found on chromosome 17.
6. ENPP1 This gene is a member of the ecto-nucleotide pyrophosphatase/phosphodiesterase (ENPP) family. The encoded protein is a type II transmembrane glycoprotein comprising two identical disulfide-bonded subunits. This protein has broad specificity and cleaves a variety of substrates, including phosphodiester bonds of nucleotides and nucleotide sugars and pyrophosphate bonds of nucleotides and nucleotide sugars. This protein may function to hydrolyze nucleoside 5′ triphosphates to their corresponding monophosphates and may also hydrolyze diadenosine polyphosphates. Mutations in this gene have been associated with ‘idiopathic’ infantile arterial calcification, ossification of the posterior longitudinal ligament of the spine (OPLL), and insulin resistance.
7. ENTPD6 ENTPD6 is similar to E-type nucleotidases (NTPases). NTPases, such as CD39, mediate catabolism of extracellular nucleotides. ENTPD6 contains 4 apyrase-conserved regions which are characteristic of NTPases. Alternative splicing results in multiple transcript variants encoding different isoforms.
8. ENTPD5 The protein encoded by this gene is similar to E-type nucleotidases (NTPases)/ecto-ATPase/apyrases. NTPases, such as CD39, mediate catabolism of extracellular nucleotides. ENTPD5 contains 4 apyrase-conserved regions which is characteristic of NTPases.
9. PKM This gene encodes a protein involved in glycolysis. The encoded protein is a pyruvate kinase that catalyzes the transfer of a phosphoryl group from phosphoenolpyruvate to ADP, generating ATP and pyruvate. This protein has been shown to interact with thyroid hormone and may mediate cellular metabolic effects induced by thyroid hormones. This protein has been found to bind Opa protein, a bacterial outer membrane protein involved in gonococcal adherence to an invasion of human cells, suggesting a role of this protein in bacterial pathogenesis. Several alternatively spliced transcript variants encoding a few distinct isoforms have been reported.
10. UMPS This gene encodes a uridine 5′-monophosphate synthase. The encoded protein is a bifunctional enzyme that catalyzes the final two steps of the de novo pyrimidine biosynthetic pathway. The first reaction is carried out by the N-terminal enzyme orotate phosphoribosyltransferase which converts orotic acid to orotidine-5′-monophosphate. The terminal reaction is carried out by the C-terminal enzyme OMP decarboxylase which converts orotidine-5′-monophosphate to uridine monophosphate. Defects in this gene are the cause of hereditary orotic aciduria. Alternate splicing results in multiple transcript variants.
Accordingly, in embodiments, Somahol can be administered to modulate ENTPD4 levels and/or activity. In embodiments, functional partners of ENTPD4 include one or more of ADSSL1, ITPA, ATIC, ENPP3, ADSS, ENPP1, ENTPD6, ENTPD5, PKM and UMPS.

### OXT

Oxytocin is a neuropeptide involved in animal and human reproductive and social behavior. Oxytocin operates through both synaptic and cellular plasticity mechanisms to rewire brain circuitry to increase neuronal representation of sensory stimuli. This increased sensory salience facilitates both the formation and maintenance of complex behaviors. Three oxytocin signaling genes have been frequently implicated in human social behavior: OXT (structural gene for oxytocin), OXTR (oxytocin receptor), and CD38 (oxytocin secretion). This gene encodes a precursor protein that is processed to produce oxytocin and neurophysin I. Oxytocin is a posterior pituitary hormone which is synthesized as an inactive precursor in the hypothalamus along with its carrier protein neurophysin I. Together with neurophysin, it is packaged into neurosecretory vesicles and transported axonally to the nerve endings in the neurohypophysis, where it is either stored or secreted into the bloodstream. The precursor seems to be activated while it is being transported along the axon to the posterior pituitary. This hormone contracts smooth muscle during parturition and lactation. It is also involved in cognition, tolerance, adaptation and complex sexual and maternal behavior, as well as in the regulation of water excretion and cardiovascular functions.

Functional Partners of OXT include:
1. OXTR (Oxytocin receptor; Receptor for oxytocin) The protein encoded by this gene belongs to the G-protein coupled receptor family and acts as a receptor for oxytocin. Its activity is mediated by G proteins which activate a phosphatidylinositol-calcium second messenger system. The oxytocin-oxytocin receptor system plays an important role in the uterus during parturition.
2. NPS (Neuropeptide S) NPS modulates arousal and anxiety. It may play an important anorexigenic role (By similarity). Binds to its receptor NPSR1 with nanomolar affinity to increase intracellular calcium concentrations.
3. TRH (Pro-thyrotropin-releasing hormone) This gene encodes a member of the thyrotropin-releasing hormone family. Cleavage of the encoded proprotein releases mature thyrotropin-releasing hormone, which is a tripeptide hypothalamic regulatory hormone. The human proprotein contains six thyrotropin-releasing hormone tripeptides. Thyrotropin-releasing hormone is involved in the regulation and release of thyroid-stimulating hormone, as well as prolactin. Deficiency of this hormone has been associated with hypothalamic hypothyroidism.
4. CCK (Cholecystokinin) This peptide hormone induces gall bladder contraction and the release of pancreatic enzymes in the gut. This gene encodes a member of the gastrin/cholecystokinin family of proteins. The encoded preproprotein is proteolytically processed to generate multiple protein products, including the peptide hormones cholecystokinin-8, -12, -33, and others. The encoded peptides have been shown to regulate gastric acid secretion and food intake. A sulfated form of cholecystokinin-8 may modulate neuronal activity in the brain. Alternative splicing results in multiple transcript variants.
5. GNRH1 This gene encodes a preproprotein that is proteolytically processed to generate a peptide that is a member of the gonadotropin-releasing hormone (GnRH) family of peptides. Alternative splicing results in multiple transcript variants, at least one of which is secreted and then cleaved to generate gonadoliberin-1 and GnRH-associated peptide 1. Gonadoliberin-1 stimulates the release of luteinizing and follicle stimulating hormones, which are important for reproduction. Mutations in this gene are associated with hypogonadotropic hypogonadism.
6. AVP This gene encodes a member of the vasopressin/oxytocin family and preproprotein that is proteolytically processed to generate multiple protein products. These products include the neuropeptide hormone arginine vasopressin, and two other peptides, neurophysin 2 and copeptin. Arginine vasopressin is a posterior pituitary hormone that is synthesized in the supraoptic nucleus and paraventricular nucleus of the hypothalamus. Along with its carrier protein, neurophysin 2, it is packaged into neurosecretory vesicles and transported axonally to the nerve endings in the neurohypophysis where it is either stored or secreted into the bloodstream. The precursor is thought to be activated while it is being transported along the axon to the posterior pituitary. Arginine vasopressin acts as a growth factor by enhancing pH regulation through acid-base transport systems. It has a direct antidiuretic action on the kidney, and also causes vasoconstriction of the peripheral vessels. This hormone can contract smooth muscle during parturition and lactation. It is also involved in cognition, tolerance, adaptation and complex sexual and maternal behavior, as well as in the regulation of water excretion and cardiovascular functions. Mutations in this gene cause autosomal dominant neurohypophyseal diabetes insipidus (ADNDI). This gene is present in a gene cluster with the related gene oxytocin on chromosome 20.
7. AVPR1A The protein encoded by this gene acts as receptor for arginine vasopressin. This receptor belongs to the subfamily of G-protein coupled receptors which includes AVPR1B, V2R and OXT receptors. Its activity is mediated by G proteins which stimulate a phosphatidylinositol-calcium second messenger system. The receptor mediates cell contraction and proliferation, platelet aggregation, release of coagulation factor and glycogenolysis.
8. HCRT This gene encodes a hypothalamic neuropeptide precursor protein that gives rise to two mature neuropeptides, orexin A and orexin B, by proteolytic processing. Orexin A and orexin B, which bind to orphan G-protein coupled receptors HCRTR1 and HCRTR2, function in the regulation of sleep and arousal. This neuropeptide arrangement may also play a role in feeding behavior, metabolism, and homeostasis.
9. AVPR1B The protein encoded by this gene acts as receptor for arginine vasopressin. This receptor belongs to the subfamily of G-protein coupled receptors which includes AVPR1A, V2R and OXT receptors. Its activity is mediated by G proteins which stimulate a phosphatidylinositol-calcium second messenger system. The receptor is primarily located in the anterior pituitary, where it stimulates ACTH release. It is expressed at high levels in ACTH-secreting pituitary adenomas as well as in bronchial carcinoids responsible for the ectopic ACTH syndrome. A spliced antisense transcript of this gene has been reported but its function is not known.
10. TAC1 This gene encodes four products of the tachykinin peptide hormone family, substance P and neurokinin A, as well as the related peptides, neuropeptide K and neuropeptide gamma. These hormones are thought to function as neurotransmitters which interact with nerve receptors and smooth muscle cells. They are known to induce behavioral responses and function as vasodilators and secretagogues. Substance P is an antimicrobial peptide with antibacterial and antifungal properties. Multiple transcript variants encoding different isoforms have been found for this gene.

Accordingly, in embodiments, Somahol (or other therapeutic molecule disclosed herein) can be administered to modulate oxytocin levels and/or activity. In embodiments, functional partners of oxytocin include one or more of OXTR, NPS, TRH, CCK, GNRH1, AVP, AVPR1A, HCRT, AVPR1B and TAC1.

### Methods of Use

Somahol and analogs can be administered in various forms, depending on the disorder to be treated and the age, condition, and body weight of the patient, as is well known in the art. For example, where the compositions are to be administered orally, they may be formulated as tablets, capsules, granules, powders, or syrups; or for parenteral administration, they may be formulated as injections (intravenous, intramuscular, or subcutaneous), drop infusion preparations, or suppositories. Any suitable route or mode of administration can be employed for providing the patient with a therapeutically or prophylactically effective dose of the therapeutic peptide. Exemplary routes or modes of administration include parenteral (e.g., intravenous, intraarterial, intramuscular, subcutaneous, intratumoral), oral, topical (nasal, transdermal, intradermal or intraocular), mucosal (e.g., nasal, sublingual, buccal, rectal, vaginal), inhalation, intralymphatic, intraspinal, intracranial, intraperitoneal, intratracheal, intravesical, intrathecal, enteral, intrapulmonary, intralymphatic, intracavital, intraorbital, intracapsular and transurethral, as well as local delivery by catheter or stent.

The methods described herein can be used to treat numerous disorders related to chronic pain and central sensitization including, for example, fibromyalgia, rheumatoid arthritis, osteoarthritis, chronic arthropathy, spinal nerve compression syndromes associated with neoplasia and/or disc herniation, chronic back pain, chronic joint pain of any etiology associated with inflammation and/or structural joint abnormalities, post herpetic neuralgia, trigeminal neuralgia, chronic metabolic neuropathy associated with chronic pain, migraine, inflammatory pain, post-surgical pain syndromes including phantom limb pain, Post Traumatic Stress Disorder, Irritable Bowel Syndrome, autonomic neuropathies, arachnoiditis, Chronic Regional Pain Syndrome, Vulvodynia, and chronic pain syndrome associated with activation of central sensitization pathways.

A pharmaceutical composition comprising a therapeutic small molecule in accordance with the present disclosure can be formulated in any pharmaceutically acceptable carrier(s) or excipient(s). As used herein, the term “pharmaceutically acceptable carrier” includes any and all solvents, dispersion media, coatings, antibacterial and antifungal agents, isotonic and absorption delaying agents, and the like that are physiologically compatible. Pharmaceutical compositions can include suitable solid or gel phase carriers or excipients. Exemplary carriers or excipients include calcium carbonate, calcium phosphate, various sugars, starches, cellulose derivatives, gelatin, and polymers such as polyethylene glycols. Exemplary pharmaceutically acceptable carriers include one or more of water, saline, phosphate buffered saline, dextrose, glycerol, ethanol and the like, as well as combinations thereof. In many cases it will be preferable to include isotonic agents, for example, sugars, polyalcohols such as mannitol, sorbitol, or sodium chloride in the composition. Pharmaceutically acceptable carriers can further comprise minor amounts of auxiliary substances such as wetting or emulsifying agents, preservatives or buffers, which enhance the shelf life or effectiveness of the therapeutic agents.

The therapeutic small molecule can be incorporated into a pharmaceutical composition suitable for parenteral administration. Suitable buffers include but are not limited to, sodium succinate, sodium citrate, sodium phosphate or potassium phosphate. Sodium chloride can be used to modify the toxicity of the solution at a concentration of 0-300 mM (optimally 150 mM for a liquid dosage form). Cryoprotectants can be included for a lyophilized dosage form, principally 0-10% sucrose (optimally 0.5-1.0%). Other suitable cryoprotectants include trehalose and lactose. Bulking agents can be included for a lyophilized dosage form, principally 1-10% mannitol (optimally 2-4%). Stabilizers can be used in both liquid and lyophilized dosage forms, principally 1-50 mM L-Methionine (optimally 5-10 mM). Other suitable bulking agents include glycine, arginine, can be included as 0-0.05%>polysorbate-80 (optimally 0.005-0.01%). Additional surfactants include but are not limited to polysorbate 20 and BRIJ surfactants.

Therapeutic small molecule preparations can be lyophilized and stored as sterile powders, preferably under vacuum, and then reconstituted in bacteriostatic water (containing, for example, benzyl alcohol preservative) or in sterile water prior to injection. Pharmaceutical compositions can be formulated for parenteral administration by injection e.g., by bolus injection or continuous infusion.

The therapeutic small molecule can be administered as a preventive measure (i.e., to avoid an ailment) at one time or multiple times. Alternatively, the therapeutic small molecule is suitably administered to the patient at one time or over a series of treatments and may be administered to the patient at any time from diagnosis onwards. The therapeutic small molecule may be administered as the sole treatment or in conjunction with other drugs or therapies useful in treating the condition in question.

Certain embodiments of the invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

### Administration

A pharmaceutical composition comprising a therapeutic molecule in accordance with the present disclosure can be formulated in any pharmaceutically acceptable carrier(s) or excipient(s). As used herein, the term “pharmaceutically acceptable carrier” includes any and all solvents, dispersion media, coatings, antibacterial and antifungal agents, isotonic and absorption delaying agents, and the like that are physiologically compatible. Pharmaceutical compositions can include suitable solid or gel phase carriers or excipients. Exemplary carriers or excipients include calcium carbonate, calcium phosphate, various sugars, starches, cellulose derivatives, gelatin, and polymers such as polyethylene glycols. Exemplary pharmaceutically acceptable carriers include one or more of water, saline, phosphate buffered saline, dextrose, glycerol, ethanol and the like, as well as combinations thereof. In many cases it will be preferable to include isotonic agents, for example, sugars, polyalcohols such as mannitol, sorbitol, or sodium chloride in the composition. Pharmaceutically acceptable carriers can further comprise minor amounts of auxiliary substances such as wetting or emulsifying agents, preservatives or buffers, which enhance the shelf-life or effectiveness of the therapeutic agents.

The therapeutic agents in the pharmaceutical compositions can be formulated in a “therapeutically effective amount” or a “prophylactically effective amount.” A “therapeutically effective amount” refers to an amount effective, at dosages and for periods of time necessary, to achieve the desired therapeutic result. A therapeutically effective amount may vary depending on the condition to be treated, the severity and course of the condition, the mode of administration, whether the agent is administered for preventive or therapeutic purposes, the bioavailability of the particular agent(s), the ability of the therapeutic small molecule to elicit a desired response in the individual, previous therapy, the age, weight and sex of the patient, the patient's clinical history and response to the agent, the type of the therapeutic small molecule used, discretion of the attending physician, etc. A therapeutically effective amount is also one in which any toxic or detrimental effects is outweighed by the therapeutically beneficial effects. A “prophylactically effective amount” refers to an amount effective, at dosages and for periods of time necessary, to achieve the desired prophylactic result.

The solution containing SOMAHOL (or other therapeutic molecule disclosed herein) is suitably administered to the patient at one time or over a series of treatments and may be administered to the patient at any time from diagnosis onwards. Alternatively, it can be administered as a preventive measure (i.e., to avoid infection). The solution can be administered as the sole treatment or in conjunction with other drugs or therapies useful in treating the condition in question.

As a general proposition, a therapeutically effective amount or prophylactically effective amount of a therapeutic molecule disclosed herein will be administered in a range from about 1 ng/kg body weight to about 100 mg/kg body weight whether by one or more administrations. In a particular embodiment, each therapeutic small molecule is administered in the range of from about 1 ng/kg body weight to about 10 mg/kg body weight, about 1 ng/kg body weight to about 1 mg/kg body weight, about 1 ng/kg body weight to about 100 g/kg body weight, about 1 ng/kg body weight to about 10 g/kg body weight, about 1 ng/kg body weight/day to about 1 g/kg body weight, about 1 ng/kg body weight to about 100 ng/kg body weight, about 1 ng/kg body weight to about 10 ng/kg body weight, about 10 ng/kg body weight to about 100 mg/kg body weight, about 10 ng/kg body weight to about 10 mg/kg body weight, about 10 ng/kg body weight to about 1 mg/kg body weight, about 10 ng/kg body weight/to about 100 g/kg body weight, about 10 ng/kg body weight to about 10 mg/kg body weight, about 10 ng/kg body weight to about 1 mg/kg body weight, 10 ng/kg body weight to about 100 ng/kg body weight/, about 100 ng/kg body weight to about 100 mg/kg body weight, about 100 ng/kg body weight to about 10 mg/kg body weight, about 100 ng/kg body weight to about 1 mg/kg body weight, about 100 ng/kg body weight to about 100 mg/kg body weight, about 100 ng/kg body weight to about 10 mg/kg body weight, about 100 ng/kg body weight to about 1 mg/kg body weight, about 1 mg/kg body weight to about 100 mg/kg body weight, about 1 mg/kg body weight to about 10 mg/kg body weight/day, about 1 mg/kg body weight to about 1 mg/kg body weight, about 1 mg/kg body weight to about 100 mg/kg body weight, about 1 mg/kg body weight to about 10 mg/kg body weight, about 10 mg/kg body weight to about 100 mg/kg body weight, about 10 mg/kg body weight to about 10 mg/kg body weight, about 10 mg/kg body weight to about 1 mg/kg body weight/day, about 10 mg/kg body weight to about 100 mg/kg body weight, about 100 mg/kg body weight/day to about 100 mg/kg body weight, about 100 mg/kg body weight/day to about 10 mg/kg body weight, about 100 mg/kg body weight/day to about 1 mg/kg body weight, about 1 mg/kg body weight to about 100 mg/kg body weight, about 1 mg/kg body weight to about 10 mg/kg body weight, about 10 mg/kg body weight to about 100 mg/kg body weight/day.

In other embodiments, a therapeutic molecule disclosed herein is administered in the range of about 10 ng to about 100 ng per individual administration, about 10 ng to about 1 g per individual administration, about 10 ng to about 10 g per individual administration, about 10 ng to about 100 mg per individual administration, about 10 ng to about 1 mg per individual administration, about 10 ng to about 10 mg per individual administration, about 10 ng to about 100 mg per individual administration, about 10 ng to about 1000 mg per injection, about 10 ng to about 10,000 mg per individual administration, about 100 ng to about 1 mg per individual administration, about 100 ng to about 10 mg per individual administration, about 100 ng to about 100 mg per individual administration, about 100 ng to about 1 mg per individual administration, about 100 ng to about 10 mg per individual administration, about 100 ng to about 100 mg per individual administration, about 100 ng to about 1000 mg per injection, about 100 ng to about 10,000 mg per individual administration, about 1 mg to about 10 mg per individual administration, about 1 mg to about 100 mg per individual administration, about 1 mg to about 1 mg per individual administration, about 1 mg to about 10 mg per individual administration, about 1 mg to about 100 mg per individual administration, about 1 mg to about 1000 mg per injection, about 1 mg to about 10,000 mg per individual administration, about 10 mg to about 100 mg per individual administration, about 10 mg to about 1 mg per individual administration, about 10 mg to about 10 mg per individual administration, about 10 mg to about 100 mg per individual administration, about 10 mg to about 1000 mg per injection, about 10 mg to about 10,000 mg per individual administration, about 100 mg to about 1 mg per individual administration, about 100 mg to about 10 mg per individual administration, about 100 mg to about 100 mg per individual administration, about 100 mg to about 1000 mg per injection, about 100 mg to about 10,000 mg per individual administration, about 1 mg to about 10 mg per individual administration, about 1 mg to about 100 mg per individual administration, about 1 mg to about 1000 mg per injection, about 1 mg to about 10,000 mg per individual administration, about 10 mg to about 100 mg per individual administration, about 10 mg to about 1000 mg per injection, about 10 mg to about 10,000 mg per individual administration, about 100 mg to about 1000 mg per injection, about 100 mg to about 10,000 mg per individual administration and about 1000 mg to about 10,000 mg per individual administration. The therapeutic small molecule may be administered daily, every 2, 3, 4, 5, 6, 7 or 10 days, or every 1, 2, 3 or 4 weeks.

In other particular embodiments, the amount of the therapeutic molecule disclosed herein can be administered at a dose of about 0.0006 mg, 0.001 mg, 0.003 mg, 0.006 mg, 0.01 mg, 0.03 mg, 0.06 mg, 0.1 mg, 0.3 mg, 0.6 mg, 1 mg, 3 mg, 6 mg, 10 mg, 30 mg, 60 mg, 100 mg, 300 mg, 600 mg, 1000 mg, 2000 mg, 5000 mg or 10,000 mg. As expected, the dosage will be dependent on the condition, size, age and condition of the patient.

In other aspects of this embodiment, a pharmaceutical composition compound disclosed herein reduces signs/symptoms of an ailment such as a neurological disorder by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, a pharmaceutical composition disclosed herein reduces signs/symptoms of an ailment such as a neurological disorder from, e.g., about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

A pharmaceutical composition disclosed herein is in an amount sufficient to allow customary administration to an individual. In aspects of this embodiment, a pharmaceutical composition disclosed herein can be, e.g., at least 5 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, or at least 100 mg of a pharmaceutical composition. In other aspects of this embodiment, a pharmaceutical composition disclosed herein may be, e.g., at least 5 mg, at least 10 mg, at least 20 mg, at least 25 mg, at least 50 mg, at least 75 mg, at least 100 mg, at least 200 mg, at least 300 mg, at least 400 mg, at least 500 mg, at least 600 mg, at least 700 mg, at least 800 mg, at least 900 mg, at least 1,000 mg, at least 1,100 mg, at least 1,200 mg, at least 1,300 mg, at least 1,400 mg, or at least 1,500 mg of a pharmaceutical composition. In yet other aspects of this embodiment, a pharmaceutical composition disclosed herein may be in the range of, e.g., about 5 mg to about 100 mg, about 10 mg to about 100 mg, about 50 mg to about 150 mg, about 100 mg to about 250 mg, about 150 mg to about 350 mg, about 250 mg to about 500 mg, about 350 mg to about 600 mg, about 500 mg to about 750 mg, about 600 mg to about 900 mg, about 750 mg to about 1,000 mg, about 850 mg to about 1,200 mg, or about 1,000 mg to about 1,500 mg. In still other aspects of this embodiment, a pharmaceutical composition disclosed herein may be in the range of, e.g., about 10 mg to about 250 mg, about 10 mg to about 500 mg, about 10 mg to about 750 mg, about 10 mg to about 1,000 mg, about 10 mg to about 1,500 mg, about 50 mg to about 250 mg, about 50 mg to about 500 mg, about 50 mg to about 750 mg, about 50 mg to about 1,000 mg, about 50 mg to about 1,500 mg, about 100 mg to about 250 mg, about 100 mg to about 500 mg, about 100 mg to about 750 mg, about 100 mg to about 1,000 mg, about 100 mg to about 1,500 mg, about 200 mg to about 500 mg, about 200 mg to about 750 mg, about 200 mg to about 1,000 mg, about 200 mg to about 1,500 mg, about 5 mg to about 1,500 mg, about 5 mg to about 1,000 mg, or about 5 mg to about 250 mg.

A pharmaceutical composition disclosed herein can comprise a solvent, emulsion or other diluent in an amount sufficient to dissolve a pharmaceutical composition disclosed herein. In other aspects of this embodiment, a pharmaceutical composition disclosed herein may comprise a solvent, emulsion or a diluent in an amount of, e.g., less than about 90% (v/v), less than about 80% (v/v), less than about 70% (v/v), less than about 65% (v/v), less than about 60% (v/v), less than about 55% (v/v), less than about 50% (v/v), less than about 45% (v/v), less than about 40% (v/v), less than about 35% (v/v), less than about 30% (v/v), less than about 25% (v/v), less than about 20% (v/v), less than about 15% (v/v), less than about 10% (v/v), less than about 5% (v/v), or less than about 1% (v/v). In other aspects of this embodiment, a pharmaceutical composition disclosed herein may comprise a solvent, emulsion or other diluent in an amount in a range of, e.g., about 1% (v/v) to 90% (v/v), about 1% (v/v) to 70% (v/v), about 1% (v/v) to 60% (v/v), about 1% (v/v) to 50% (v/v), about 1% (v/v) to 40% (v/v), about 1% (v/v) to 30% (v/v), about 1% (v/v) to 20% (v/v), about 1% (v/v) to 10% (v/v), about 2% (v/v) to 50% (v/v), about 2% (v/v) to 40% (v/v), about 2% (v/v) to 30% (v/v), about 2% (v/v) to 20% (v/v), about 2% (v/v) to 10% (v/v), about 4% (v/v) to 50% (v/v), about 4% (v/v) to 40% (v/v), about 4% (v/v) to 30% (v/v), about 4% (v/v) to 20% (v/v), about 4% (v/v) to 10% (v/v), about 6% (v/v) to 50% (v/v), about 6% (v/v) to 40% (v/v), about 6% (v/v) to 30% (v/v), about 6% (v/v) to 20% (v/v), about 6% (v/v) to 10% (v/v), about 8% (v/v) to 50% (v/v), about 8% (v/v) to 40% (v/v), about 8% (v/v) to 30% (v/v), about 8% (v/v) to 20% (v/v), about 8% (v/v) to 15% (v/v), or about 8% (v/v) to 12% (v/v).

The final concentration of a pharmaceutical composition disclosed herein in a pharmaceutical composition disclosed herein can be of any concentration desired. In an aspect of this embodiment, the final concentration of a pharmaceutical composition in a pharmaceutical composition may be a therapeutically effective amount. In other aspects of this embodiment, the final concentration of a pharmaceutical composition in a pharmaceutical composition may be, e.g., at least 0.00001 mg/mL, at least 0.0001 mg/mL, at least 0.001 mg/mL, at least 0.01 mg/mL, at least 0.1 mg/mL, at least 1 mg/mL, at least 10 mg/mL, at least 25 mg/mL, at least 50 mg/mL, at least 100 mg/mL, at least 200 mg/mL or at least 500 mg/mL. In other aspects of this embodiment, the final concentration of a pharmaceutical composition in a pharmaceutical composition may be in a range of, e.g., about 0.00001 mg/mL to about 3,000 mg/mL, about 0.0001 mg/mL to about 3,000 mg/mL, about 0.01 mg/mL to about 3,000 mg/mL, about 0.1 mg/mL to about 3,000 mg/mL, about 1 mg/mL to about 3,000 mg/mL, about 250 mg/mL to about 3,000 mg/mL, about 500 mg/mL to about 3,000 mg/mL, about 750 mg/mL to about 3,000 mg/mL, about 1,000 mg/mL to about 3,000 mg/mL, about 100 mg/mL to about 2,000 mg/mL, about 250 mg/mL to about 2,000 mg/mL, about 500 mg/mL to about 2,000 mg/mL, about 750 mg/mL to about 2,000 mg/mL, about 1,000 mg/mL to about 2,000 mg/mL, about 100 mg/mL to about 1,500 mg/mL, about 250 mg/mL to about 1,500 mg/mL, about 500 mg/mL to about 1,500 mg/mL, about 750 mg/mL to about 1,500 mg/mL, about 1,000 mg/mL to about 1,500 mg/mL, about 100 mg/mL to about 1,200 mg/mL, about 250 mg/mL to about 1,200 mg/mL, about 500 mg/mL to about 1,200 mg/mL, about 750 mg/mL to about 1,200 mg/mL, about 1,000 mg/mL to about 1,200 mg/mL, about 100 mg/mL to about 1,000 mg/mL, about 250 mg/mL to about 1,000 mg/mL, about 500 mg/mL to about 1,000 mg/mL, about 750 mg/mL to about 1,000 mg/mL, about 100 mg/mL to about 750 mg/mL, about 250 mg/mL to about 750 mg/mL, about 500 mg/mL to about 750 mg/mL, about 100 mg/mL to about 500 mg/mL, about 250 mg/mL to about 500 mg/mL, about 0.00001 mg/mL to about 0.0001 mg/mL, about 0.00001 mg/mL to about 0.001 mg/mL, about 0.00001 mg/mL to about 0.01 mg/mL, about 0.00001 mg/mL to about 0.1 mg/mL, about 0.00001 mg/mL to about 1 mg/mL, about 0.001 mg/mL to about 0.01 mg/mL, about 0.001 mg/mL to about 0.1 mg/mL, about 0.001 mg/mL to about 1 mg/mL, about 0.001 mg/mL to about 10 mg/mL, or about 0.001 mg/mL to about 100 mg/mL.

Aspects of the present specification disclose, in part, treating an individual who is susceptible to an ailment (e.g., a neurological disorder) or addiction or suffering from an ailment or addiction. As used herein, the term “treating,” refers to reducing or eliminating the signs/symptoms of the ailment; or lowering or depleting signs/symptoms. For example, the term “treating” can mean reducing a symptom of a condition characterized by an ailment, by, e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 100%. Those of skill in the art will know the appropriate symptoms or indicators associated with a specific type of ailment and will know how to determine if an individual is a candidate for treatment as disclosed herein.

In aspects of this embodiment, a therapeutically effective amount of a pharmaceutical composition disclosed herein reduces signs/symptoms of an ailment, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a pharmaceutical composition disclosed herein reduces signs/symptoms of an ailment by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a pharmaceutical composition disclosed herein reduces signs/symptoms of an ailment by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In yet other aspects of this embodiment, a therapeutically effective amount of a pharmaceutical composition disclosed herein generally is in the range of about 0.001 mg/kg to about 100 mg/kg and administered, for example, every 3, 5, 7, 10 or 14 days. In aspects of this embodiment, an effective amount of a pharmaceutical composition disclosed herein may be, e.g., at least 0.001 mg/kg, at least 0.01 mg/kg, at least 0.1 mg/kg, at least 1.0 mg/kg, at least 5.0 mg/kg, at least 10 mg/kg, at least 15 mg/kg, at least 20 mg/kg, at least 25 mg/kg, at least 30 mg/kg, at least 35 mg/kg, at least 40 mg/kg, at least 45 mg/kg, or at least 50 mg/kg and administered, for example, every 3, 5, 7, 10 or 14 days. In other aspects of this embodiment, an effective amount of a pharmaceutical composition disclosed herein may be in the range of, e.g., about 0.001 mg/kg to about 10 mg/kg, about 0.001 mg/kg/day to about 15 mg/kg, about 0.001 mg/kg to about 20 mg/kg, about 0.001 mg/kg to about 25 mg/kg, about 0.001 mg/kg to about 30 mg/kg, about 0.001 mg/kg to about 35 mg/kg, about 0.001 mg/kg to about 40 mg/kg, about 0.001 mg/kg to about 45 mg/kg, about 0.001 mg/kg to about 50 mg/kg, about 0.001 mg/kg to about 75 mg/kg, or about 0.001 mg/kg to about 100 mg/kg and administered, for example, every 3, 5, 7, 10 or 14 days. In yet other aspects of this embodiment, an effective amount of a pharmaceutical composition disclosed herein may be in the range of, e.g., about 0.01 mg/kg to about 10 mg/kg, about 0.01 mg/kg to about 15 mg/kg, about 0.01 mg/kg to about 20 mg/kg, about 0.01 mg/kg to about 25 mg/kg, about 0.01 mg/kg to about 30 mg/kg, about 0.01 mg/kg to about 35 mg/kg, about 0.01 mg/kg to about 40 mg/kg, about 0.01 mg/kg to about 45 mg/kg, about 0.01 mg/kg to about 50 mg/kg, about 0.01 mg/kg to about 75 mg/kg, or about 0.01 mg/kg to about 100 mg/kg and administered, for example, every 3, 5, 7, 10 or 14 days. In still other aspects of this embodiment, an effective amount of a pharmaceutical composition disclosed herein may be in the range of, e.g., about 0.1 mg/kg to about 10 mg/kg, about 0.1 mg/kg to about 15 mg/kg, about 0.1 mg/kg to about 20 mg/kg, about 0.1 mg/kg to about 25 mg/kg, about 0.1 mg/kg to about 30 mg/kg, about 0.1 mg/kg to about 35 mg/kg, about 0.1 mg/kg to about 40 mg/kg, about 0.1 mg/kg to about 45 mg/kg, about 0.1 mg/kg to about 50 mg/kg, about 0.1 mg/kg to about 75 mg/kg, or about 0.1 mg/kg to about 100 mg/kg and administered, for example, every 3, 5, 7, 10 or 14 days.

Dosing can be single dosage or cumulative (serial dosing), and can be readily determined by one skilled in the art. For example, treatment of an ailment or addiction infection can comprise a one-time administration of an effective dose of a pharmaceutical composition disclosed herein. Alternatively, treatment of an ailment or addiction may include multiple administrations of an effective dose of a pharmaceutical composition carried out over a range of time periods, such as, e.g., once daily, twice daily, trice daily, once every few days, or once weekly. The timing of administration can vary from individual to individual, depending upon such factors as the severity of an individual's symptoms. For example, an effective dose of a pharmaceutical composition disclosed herein can be administered to an individual once daily for an indefinite period of time, or until the individual no longer requires therapy. A person of ordinary skill in the art will recognize that the condition of the individual can be monitored throughout the course of treatment and that the effective amount of a pharmaceutical composition disclosed herein that is administered can be adjusted accordingly.

In one embodiment, a therapeutic disclosed herein is capable of reducing the signs/symptoms of an ailment such as a neurological disorder by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects of this embodiment, the signs/symptoms of an ailment such as a neurological disorder are decreased by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In a further embodiment, the therapeutic small molecule and its derivatives have half-lives of 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 1 week, 2 weeks, 3 weeks, 4 weeks, one month, two months, three months, four months or more.

In an embodiment, the period of administration of a therapeutic molecule disclosed herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In aspects of this embodiment, a therapeutically effective amount of a therapeutic disclosed herein reduces the signs/symptoms of an ailment such as a neurological disorder by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a therapeutic disclosed herein reduces the signs/symptoms of an ailment such as a neurological disorder by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a therapeutic disclosed herein reduces the signs/symptoms of an ailment such as a neurological disorder, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In an embodiment, treatment with a therapeutic molecule disclosed herein decreases the recovery time of an ailment such as a neurological disorder, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In an embodiment, treatment with a therapeutic molecule disclosed herein decreases the signs/symptoms of addiction recovery such as withdrawal symptoms, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In an embodiment, treatment with a therapeutic molecule disclosed herein increases neuroplasticity and/or improves neural function in a patient, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In one embodiment, the dose of the composition may be administered daily, semi-weekly, weekly, bi-weekly, or monthly. The period of treatment may be for a week, two weeks, a month, two months, four months, six months, eight months, a year, or longer. The initial dose may be larger than a sustaining dose. In one embodiment, the dose ranges from a weekly dose of at least 0.01 mg/kg, at least 0.25 mg/kg, at least 0.3 mg/kg, at least 0.5 mg/kg, at least 0.75 mg/kg, at least 1 mg/kg, at least 2 mg/kg, at least 3 mg/kg, at least 4 mg/kg, at least 5 mg/kg, at least 6 mg/kg, at least 7 mg/kg, at least 8 mg/kg, at least 9 mg/kg, at least 10 mg/kg, at least 15 mg/kg, at least 20 mg/kg, at least 25 mg/kg, or at least 30 mg/kg In one embodiment, a weekly dose may be at most 1.5 mg/kg, at most 2 mg/kg, at most 2.5 mg/kg, at most 3 mg/kg, at most 4 mg/kg, at most 5 mg/kg, at most 6 mg/kg, at most 7 mg/kg, at most 8 mg/kg, at most 9 mg/kg, at most 10 mg/kg, at most 15 mg/kg, at most 20 mg/kg, at most 25 mg/kg, or at most 30 mg/kg. In a particular aspect, the weekly dose may range from 5 mg/kg to 20 mg/kg. In an alternative aspect, the weekly dose may range from 10 mg/kg to 15 mg/kg.

The present specification also provides a pharmaceutical composition for the administration to a subject. The pharmaceutical composition disclosed herein may further include a pharmaceutically acceptable carrier, excipient, or diluent. As used herein, the term “pharmaceutically acceptable” means that the composition is sufficient to achieve the therapeutic effects without deleterious side effects, and may be readily determined depending on the type of the diseases, the patient's age, body weight, health conditions, gender, and drug sensitivity, administration route, administration mode, administration frequency, duration of treatment, drugs used in combination or coincident with the composition disclosed herein, and other factors known in medicine.

The pharmaceutical composition herein may further include a pharmaceutically acceptable carrier. For oral administration, the carrier may include, but is not limited to, a binder, a lubricant, a disintegrant, an excipient, a solubilizer, a dispersing agent, a stabilizer, a suspending agent, a colorant, and a flavorant. For injectable preparations, the carrier may include a buffering agent, a preserving agent, an analgesic, a solubilizer, an isotonic agent, and a stabilizer. For preparations for topical administration, the carrier may include a base, an excipient, a lubricant, and a preserving agent.

The disclosed compositions may be formulated into a variety of dosage forms in combination with the aforementioned pharmaceutically acceptable carriers. For example, for oral administration, the pharmaceutical composition may be formulated into tablets, troches, capsules, elixirs, suspensions, syrups or wafers. For injectable preparations, the pharmaceutical composition may be formulated into an ampule as a single dosage form or a multidose container. The pharmaceutical composition may also be formulated into solutions, suspensions, tablets, pills, capsules and long-acting preparations.

On the other hand, examples of the carrier, the excipient, and the diluent suitable for the pharmaceutical formulations include, without limitation, lactose, dextrose, sucrose, sorbitol, mannitol, xylitol, erythritol, maltitol, starch, acacia rubber, alginate, gelatin, calcium phosphate, calcium silicate, cellulose, methylcellulose, microcrystalline cellulose, polyvinylpyrrolidone, water, methylhydroxybenzoate, propylhydroxybenzoate, talc, magnesium stearate and mineral oils. In addition, the pharmaceutical formulations may further include fillers, anti-coagulating agents, lubricants, humectants, flavorants, and antiseptics.

Further, the pharmaceutical composition disclosed herein may have any formulation selected from the group consisting of tablets, pills, powders, granules, capsules, suspensions, liquids for internal use, emulsions, syrups, sterile aqueous solutions, non-aqueous solvents, lyophilized formulations and suppositories.

The composition may be formulated into a single dosage form suitable for the patient's body, and preferably is formulated into a preparation useful for small molecule drugs according to the typical method in the pharmaceutical field so as to be administered by an oral or parenteral route such as through skin, intravenous, intramuscular, intra-arterial, intramedullary, intramedullary, intraventricular, pulmonary, transdermal, subcutaneous, intraperitoneal, intranasal, intracolonic, topical, sublingual, vaginal, or rectal administration, but is not limited thereto.

The composition may be used by blending with a variety of pharmaceutically acceptable carriers such as physiological saline or organic solvents. In order to increase the stability or absorptivity, carbohydrates such as glucose, sucrose or dextrans, antioxidants such as ascorbic acid or glutathione, chelating agents, low molecular weight proteins or other stabilizers may be used.

The administration dose and frequency of the pharmaceutical composition disclosed herein are determined by the type of active ingredient, together with various factors such as the disease to be treated, administration route, patient's age, gender, and body weight, and disease severity.

The total effective dose of the compositions disclosed herein may be administered to a patient in a single dose or may be administered for a long period of time in multiple doses according to a fractionated treatment protocol. In the pharmaceutical composition disclosed herein, the content of active ingredient may vary depending on the disease severity. Preferably, the total daily dose of the small molecule disclosed herein may be approximately 0.0001 μg to 500 mg per 1 kg of body weight of a patient. However, the effective dose of the small molecule is determined considering various factors including patient's age, body weight, health conditions, gender, disease severity, diet, and secretion rate, in addition to administration route and treatment frequency of the pharmaceutical composition. In view of this, those skilled in the art may easily determine an effective dose suitable for the particular use of the pharmaceutical composition disclosed herein. The pharmaceutical composition disclosed herein is not particularly limited to the formulation, and administration route and mode, as long as it shows suitable effects.

Moreover, the pharmaceutical composition may be administered alone or in combination or coincident with other pharmaceutical formulations showing prophylactic or therapeutic efficacy.

In various embodiments, a formulation can include, without limitation, combinations of bioactive agents (such as viruses, proteins, antibodies, peptides and the like as described herein) in the formulation. For example, a formulation as described herein can include a single bioactive agent for treatment of one or more conditions, including without limitation, disease. A formulation as described herein also can include, in an embodiment, without limitation, two or more different bioactive agents for a single or multiple conditions. Use of multiple bioactive agents in a formulation can be directed to, for example, the same or different indications. Similarly, in another embodiment, multiple bioactive agents can be used in a formulation to treat, for example, both a pathological condition and one or more side effects caused by the primary treatment. In a further embodiment, multiple bioactive agents also can be included, without limitation, in a formulation as described herein to accomplish different medical purposes including, for example, simultaneous treatment and monitoring of the progression of the pathological condition. In an additional embodiment, multiple, concurrent therapies such as those exemplified herein as well as other combinations well known in the art are particularly useful for patient compliance because a single formulation can be sufficient for some or all suggested treatments and/or diagnosis. Those skilled in the art will know those bioactive agents that can be admixed for a wide range of combination therapies. Similarly, in various embodiments, a formulation can be used with a small molecule drug and combinations of one or more bioactive agents together with one or more small molecule pharmaceuticals. Therefore, in various embodiments a formulation is provided containing 1, 2, 3, 4, 5 or 6 or more different bioactive agents, as well as, for one or more bioactive agents combined with one or more small molecule pharmaceuticals.

In various embodiments, a formulation can include, one or more preservatives and/or additives known in the art. Similarly, a formulation can further be formulated, without limitation, into any of various known delivery formulations. For example, in an embodiment, a formulation can include, surfactants, adjuvant, biodegradable polymers, hydrogels, etc., such optional components, their chemical and functional characteristics are known in the art. Similarly known in the art are formulations that facilitate rapid, sustained or delayed release of the bioactive agents after administration. A formulation as described can be produced to include these or other formulation components known in the art.

The composition can therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages may be ascertained through use of appropriate dose-response data. In various embodiments, the bioactive agents in formulations described herein can, without limitation, be administered to patients throughout an extended time period, such as chronic administration for a chronic condition. The composition can be a solid, a semi-solid or an aerosol and a pharmaceutical compositions is formulated as a tablet, geltab, lozenge, orally dissolved strip, capsule, syrup, oral suspension, emulsion, granule, sprinkle or pellet.

In an embodiment, for oral, rectal, vaginal, parenteral, pulmonary, sublingual and/or intranasal delivery formulations, tablets can be made by compression or molding, optionally with one or more accessory ingredients or additives. In an embodiment, compressed tablets are prepared, for example, by compressing in a suitable tableting machine, the active ingredients in a free-flowing form such as a powder or granules, optionally mixed with a binder (for example, without limitation, povidone, gelatin, hydroxypropylmethyl cellulose), lubricant, inert diluent, preservative, disintegrant (for example, without limitation, sodium starch glycolate, cross-linked povidone, cross-linked sodium carboxymethyl cellulose) and/or surface-active or dispersing agent.

In an embodiment, molded tablets are made, for example, without limitation, by molding in a suitable tableting machine, a mixture of powdered compounds moistened with an inert liquid diluent. In an embodiment, the tablets may optionally be coated or scored, and may be formulated so as to provide slow or controlled release of the active ingredients, using, for example, without limitation, hydroxypropylmethyl cellulose in varying proportions to provide the desired release profile. In an embodiment, tablets may optionally be provided with a coating, without limitation, such as a thin film, sugar coating, or an enteric coating to provide release in parts of the gut other than the stomach. In an embodiment, processes, equipment, and toll manufacturers for tablet and capsule making are well-known in the art.

In an embodiment, capsule formulations can utilize either hard or soft capsules, including, without limitation, gelatin capsules or vegetarian capsules such as those made out of hydroxymethylpropylcellulose (HMPC). In an embodiment, a type of capsule is a gelatin capsule. In an embodiment, capsules may be filled using a capsule filling machine such as, without limitation, those available from commercial suppliers such as Miranda International or employing capsule manufacturing techniques well-known in the industry, as described in detail in Pharmaceutical Capules, 2.sup.nd Ed., F. Podczeck and B. Jones, 2004. In an embodiment, capsule formulations may be prepared, without limitation, using a toll manufacturing center such as the Chao Center for Industrial Pharmacy & Contract Manufacturing, located at Purdue Research Park.

Packaging and instruments for administration may be determined by a variety of considerations, such as, without limitation, the volume of material to be administered, the conditions for storage, whether skilled healthcare practitioners will administer or patient self-compliance, the dosage regime, the geopolitical environment (e.g., exposure to extreme conditions of temperature for developing nations), and other practical considerations.

Injection devices include pen injectors, auto injectors, safety syringes, injection pumps, infusion pumps, glass prefilled syringes, plastic prefilled syringes and needle free injectors syringes may be prefilled with liquid, or may be dual chambered, for example, for use with lyophilized material. An example of a syringe for such use is the Lyo-Ject™, a dual-chamber pre-filled lyosyringe available from Vetter GmbH, Ravensburg, Germany. Another example is the LyoTip which is a prefilled syringe designed to conveniently deliver lyophilized formulations available from LyoTip, Inc., Camarillo, California, U.S.A. Administration by injection may be, without limitation intravenous, intramuscular, intraperitoneal, or subcutaneous, as appropriate. Administrations by non-injection route may be, without limitation, nasal, oral, cocular, dermal, or pulmonary, as appropriate.

In certain embodiments, kits can include one or more single or multi-chambered syringes (e.g., liquid syringes and lyosyringes) for administering one or more formulations described herein. In various embodiments, the kit can comprise formulation components for parenteral, subcutaneous, intramuscular or IV administration, sealed in a vial under partial vacuum in a form ready for loading into a syringe and administration to a subject. In this regard, the composition can be disposed therein under partial vacuum. In all of these embodiments and others, the kits can contain one or more vials in accordance with any of the foregoing, wherein each vial contains a single unit dose for administration to a subject.

The kits can comprise lyophilates, disposed as herein, that upon reconstitution provide compositions in accordance therewith. In various embodiment the kits can contain a lyophilate and a sterile diluent for reconstituting the lyophilate.

Also described herein, are methods for treating a subject in need of therapy, comprising administering to the subject an effective amount of a formulation as described herein. The therapeutically effective amount or dose of a formulation will depend on the disease or condition of the subject and actual clinical setting.

In an embodiment, a formulation as described herein can be administered by any suitable route, specifically by parenteral (including subcutaneous, intramuscular, intravenous and intradermal) administration. It will also be appreciated that the preferred route will vary with the condition and age of the recipient, and the disease being treated. Methods of determining the most effective means and dosage of administration are known to those of skill in the art and will vary, without limitation, with the composition used for therapy, the purpose of the therapy, and the subject being treated. Single or multiple administrations can be carried out, without limitation, the dose level and pattern being selected by the treating physician. Suitable dosage formulations and methods of administering the agents are known in the art.

The formulations as described herein can be used in the manufacture of medicaments and for the treatment of humans and other animals by administration in accordance with conventional procedures.

Also provided herein are combinatorial methods for developing suitable virus formulations using combinations of amino acids. These methods are effective for developing stable liquid or lyophilized formulations, and particularly pharmaceutical virus formulations.

Compositions in accordance with embodiments described herein have desirable properties, such as desirable solubility, viscosity, syringeability and stability. Lyophilates in accordance with embodiments described herein have desirable properties, as well, such as desirable recovery, stability and reconstitution.

In an embodiment, the pH of the pharmaceutical formulation is at least about 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, or 9.

In an embodiment, the pH of the pharmaceutical formulation is from about 3 to about 9, about 4 to about 19, about 5 to about 9, about 6 to about 8, about 6 to about 7, about 6 to about 9, about 5 to about 6, about 5 to about 7, about 5 to about 8, about 4 to about 9, about 4 to about 8, about 4 to about 7, about 4 to about 6, about 4 to about 5, about 3 to about 8, about 3 to about 7, about 3 to about 6, about 3 to about 5, about 3 to about 4, about 7 to about 8, about 7 to about 9, about 7 to about 10.

### EXAMPLES

The following non-limiting examples are provided for illustrative purposes only in order to facilitate a more complete understanding of representative embodiments now contemplated. These examples are intended to be a mere subset of all possible contexts in which the components of the formulation may be combined. Thus, these examples should not be construed to limit any of the embodiments described in the present specification, including those pertaining to the type and amounts of components of the formulation and/or methods and uses thereof.

### Example 1

### Rodent Model for PTSD Assessment

Recent studies have demonstrated that rodent models can simulate stress induction and disorder development. See, for example, Verbitsky et al. Translational Psychiatry (2020) 10:132. Physical stressors used to develop PTSD models include electric shock, underwater trauma, restraint/immobilization stress, and single prolonged stress. By manipulating stress type, intensity, duration, and frequency, preclinical models reflect core PTSD phenotypes, measured through various behavioral assays. There are two conventional medications indicated for PTSD treatment, sertraline and paroxetine.

In this example, rats are exposed to various stresses (e.g., electric shock, restraint/immobilization stress and underwater trauma). Rats are then treated with Somahol and tested for signs/symptoms of PTSD. The treated population of rats is compared with a control group.

The results demonstrated that rats treated with Somahol (or other therapeutic molecule disclosed herein) recover from PTSD faster than those in the control group.

### Example 2

### Rodent Model for Addiction Assessment

Two of the fundamental features of drug addiction in humans (i.e., loss of control over use and the resulting excessive or compulsive use of the drug) have been modeled in animals using several different methods. A simple method of evaluating acquisition is to give an animal access to a drug during a daily experimental session, with deliveries available contingent upon an operant response (that is, lever press). Accordingly, this example relates to animal models effective for studying addiction. Recent studies have demonstrated that rodent models can simulate substance abuse and addiction. See, for example, Linch et al. Comparative Medicine (2010) Vol. 60, No. 3.

Rats are subject to rodent intravenous drug self-administration paradigm. Specifically, rats are implanted with chronic, indwelling catheters in the jugular vein. The catheter exits the rat on the dorsum, where it is connected to a tether-and-tubing system that is attached to a drug-loaded syringe. Responding on the active lever leads to infusions of the drug.

Acquisition of drug self-administration then is measured as the number of sessions needed to reach a criterion level of intake, which can be standardized and adjusted for dose and drug availability. The ratio of active to inactive lever press responses is used in conjugation with the intake criteria. All of the study animals are included in the analysis, regardless of whether they acquire self-administration, and the focus is on how rapidly acquisition of self-administration takes place and the percentage of animals in each group that acquire drug self-administration.

As noted above, rats are exposed to an opioid drug. Two populations of rats are exposed: a first population is then treated with Somahol and a second population (i.e., control) is left untreated or injected with saline. The treated population of rats is compared with the control group. Specifically, the ratio of active to inactive lever press responses is compared between the populations. The results demonstrate lower rates of level press responses in rats treated with Somahol. The results suggest that Somahol (or other therapeutic molecule disclosed herein) prevents addiction and/or aid in recovery from addiction.

### Example 3

### Animal Model for TBI

The next example relates to animal models effective for studying traumatic brain injury (TBI). Recent studies have demonstrated that rodent models can simulate substance abuse and addition. See, for example, Xiong et al. Nat Rev Neurosci. 2013 February; 14 (2): 128-142. doi:10.1038/nrn3407. Animal TBI models include:
Fluid percussion injury models
Controlled cortical impact injury model
Penetrating ballistic-like brain injury models
Weight drop TBI models.
Models of blast TBI
Mild TBI models

In this example, a fluid percussion injury model (FPI) is used to study the effect of Somahol on rats after FPI. In FPI models, the insult is inflicted by a pendulum striking the piston of a reservoir of fluid to generate a fluid pressure pulse to the intact dura through a craniotomy, which is made either centrally around the midline. In rats, LFPI produces a combination of focal cortical contusion and diffuse subcortical (such as hippocampus and thalamus) neuronal injury, which occurs within minutes of the impact, progresses to a loss of neurons by 12 h, and does not markedly expand into other brain regions by 7 days post injury. The contused cortex beneath the injury site enlarges over weeks to become a cavity lined with glia and continues to expand up to one year post-injury due to ongoing cell death. Over days to months, progressive degenerative cascades persist in selectively vulnerable brain regions, including the ipsilateral hippocampus, thalamus, medial septum, striatum and amygdala. LFPI produces neurobehavioural and cognitive deficits such as difficulties with movement and memory that are commonly seen in patients with TBI. Cognitive dysfunction and neurological impairments persist for more than a year following severe LFPI.

This approach produced acute and chronic TBI features similar to that observed in the LFPI literature, as quantified by histological changes, structural changes seen on MRI and chronic behavioural sequelae.

In this example, rats are subject to inflicted by a pendulum striking. Rats are then treated with Somahol and tested for signs/symptoms of TBI. The treated population of rats is compared with a control group. The results demonstrated that rats treated with Somahol (or other therapeutic molecule disclosed herein) recover from TBI faster than those in a control group.

### Example 4

### Animal Model for Multiple Sclerosis

The next example relates to animal models effective for studying Multiple Sclerosis (MS). The three most characterized animal models of MS are (1) the experimental autoimmune/allergic encephalomyelitis (EAE); (2) the virally induced chronic demyelinating disease, known as Theiler's murine encephalomyelitis virus (TMEV) infection and (3) the toxin-induced demyelination. EAE is the model which better reflects the autoimmune pathogenesis of MS and is useful to study potential experimental treatments MS is a chronic, immune-mediated, inflammatory disorder of the CNS (see, e.g., Frohman et al., 2006). The most studied animal model of MS is the experimental autoimmune encephalomyelitis (EAE), in which autoimmunity to CNS components is induced in susceptible mice through immunization with self-antigens derived from basic myelin protein.

In this example, EAE is induced in mice with through either active immunization with protein or peptide, or by passive transfer of encephalitogenic T cells. In all cases, the relevant immunogen is derived from self-CNS proteins such as myelin basic protein (MBP), proteolipid protein (PLP) or myelin oligodendrocyte glycoprotein (MOG). Immunization of SJL/J mice with the immunodominant epitope of PLP (PLP139-151) induces a relapsing-remitting (RR) disease course (see, e.g., Tuohy et al., 1989), while disease induced by the immunodominant MOG35-55 peptide in C57BL6/J mice is of chronic nature.

In this example, rats are subject EAE is induced in two populations of rats. The first population also treated with Somahol (or other therapeutic molecule disclosed herein) and the second population (i.e., control) is left untreated or injected with saline. The treated population of rats is compared with the control group for signs/symptoms of MS (e.g., inflammation). The results demonstrated that rats treated with Somahol display less sever signs/symptoms of MS. This suggests that Somahol (or other therapeutic molecule disclosed herein) confers resistance to autoimmunity and related disorders.

### Example 5

### 5-HT2A Human Serotonin GPCR Cell Based Agonist Arrestin Assay

This example was conducted to evaluate the potency (EC50) and efficacy (Max response) of compounds for the human 5-HT2A receptor in stably transfected U2OS cells determined in a GPCR cell-based assay.

To start, cells were seeded in a total volume of 20 μL into white walled, 384-well microplates and incubated at 37° C. overnight prior to testing. For agonist determination, cells were incubated with sample to induce response. Intermediate dilution of sample stocks was performed to generate 5× sample in assay buffer. 5 μL of 5× sample was added to cells and incubated at 37° C. for 120 minutes. Final assay vehicle concentration was 1%. The results are expressed as a percent efficacy relative to the maximum response of the control ligand. The assay volume was 20 μl in a 384-well plate. The compound was added in an amount of 5 μl of 5× compound that were diluted 100× in solvent if the compound was originally in DMSO. The assay was run such that the maximum tolerable DMSO concentration was 1%. The assay was run at 37° C. for 120 minutes. The reference agonist was Serotonin.

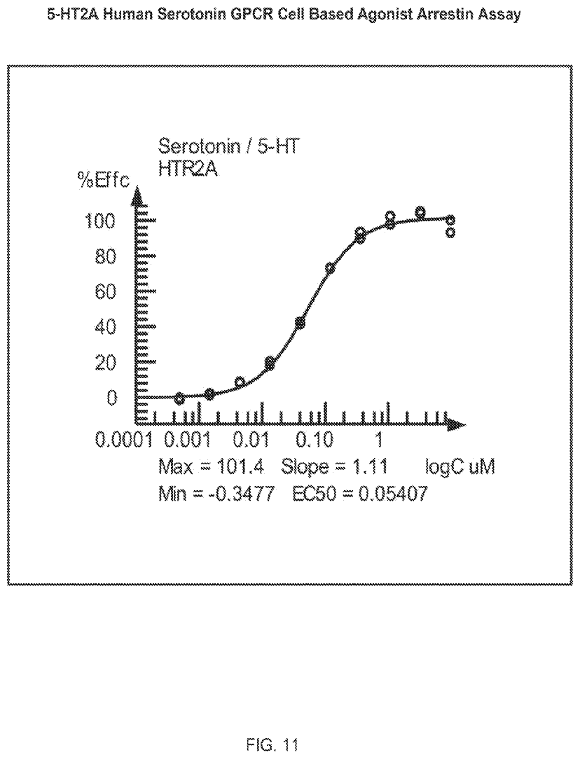

The results are shown in FIG. 11. The amino acid sequence of the 5-HT2A receptor external protein is SEQ ID NO: 1:

```
Amino-acid sequence (extracellular portion 1-75,
133-148, 216-233, 347-362 → PDZ domain 469-471 →
agonism also shown at 155-160, 336-340)
                10 20 30 40
MDILCEENTS LSSTTNSLMQ LNDDTRLYSN DFNSGEANTS
                50 60 70 80
DAFNWTVDSE NRTNLSCEGC LSPSCLSLLH LQEKNWSALL
                90 100 110 120
TAVVIILTIA GNILVIMAVS LEKKLQNATN YFLMSLAIAD
              130 140 150 160
MLLGFLVMPV SMLTILYGYR WPLPSKLCAV WIYLDVLFST
              170 180 190 200
ASIMHLCAIS LDRYVAIQNP IHHSRFNSRT KAFLKIIAVW
              210 220 230 240
TISVGISMPI PVFGLQDDSK VFKEGSCLLA DDNFVLIGSF
              250 260 270 280
VSFFIPLTIM VITYFLTIKS LQKEATLCVS DLGTRAKLAS
              290 300 310 320
FSFLPQSSLS SEKLFQRSIH REPGSYTGRR TMQSISNEQK
              330 340 350 360
ACKVLGIVFF LFVVMWCPFF ITNIMAVICK ESCNEDVIGA
              370 380 390 400
LLNVFVWIGY LSSAVNPLVY TLFNKTYRSA FSRYIQCQYK
              410 420 430 440
ENKKPLQLIL VNTIPALAYK SSQLQMGQKK NSKQDAKTTD
              450 460 470
NDCSMVALGK QHSEEASKDN SDGVNEKVSC V
```

### Example 6

### 5-HT6 Human Serotonin GPCR Cell Based Agonist Arrestin Assay

This example was conducted to evaluate the potency (EC50) and efficacy (Max response) of compounds for the human 5-HT6 receptor in stably transfected DLD1 cells determined in a GPCR cell-based assay.

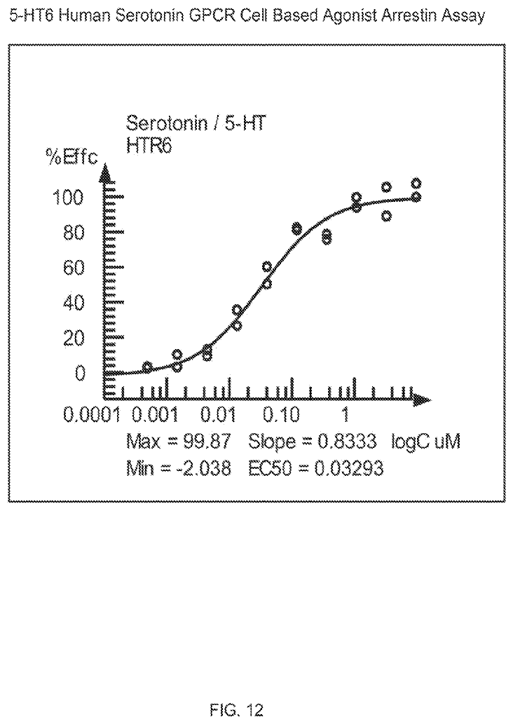

To start, cells were seeded in a total volume of 20 μL into white walled, 384-well microplates and incubated at 37° C. overnight prior to testing. For agonist determination, cells were incubated with sample to induce response. Intermediate dilution of sample stocks was performed to generate 5× sample in assay buffer. 5 μL of 5× sample was added to cells and incubated at 37° C. for 120 minutes. Final assay vehicle concentration was 1%. The results are expressed as a percent efficacy relative to the maximum response of the control ligand. The assay volume was 20 μl in a 384-well plate. The compound was added in an amount of 5 μl of 5× compound that were diluted 100× in solvent if the compound was originally in DMSO. The assay was run such that the maximum tolerable DMSO concentration was 1%. The assay was run at 37° C. for 120 minutes. The reference agonist was Serotonin/5-HT. The results are shown in FIG. 12.

### Example 7

### TRKB Human RTK Kinase Cell Based Agonist Functional Assay

This example was conducted to evaluate of the potency (EC50) and efficacy (Max response) of compounds for the human TrkB receptor in stably transfected U2OS cells determined in a PathHunter Receptor Tyrosine Kinase (RTK) cell-based assay.

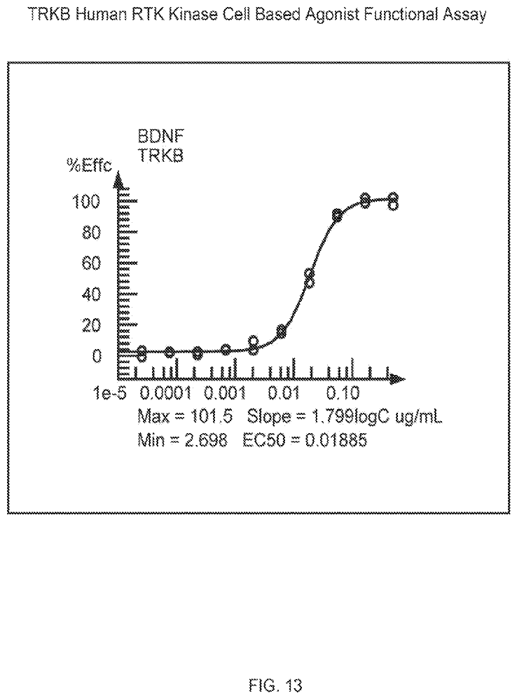

To start, cells were seeded in a total volume of 20 μL into white walled, 384-well microplates and incubated at 37° C. overnight prior to testing. For agonist determination, cells were incubated with sample to induce response. Intermediate dilution of sample stocks was performed to generate 5× sample in assay buffer. 5 μL of 5× sample was added to cells and incubated at 37° C. for 120 minutes. Final assay vehicle concentration was 1%. The results are expressed as a percent efficacy relative to the maximum response of the control ligand. The assay volume was 20 μl in a 384-well plate. The compound was added in an amount of 5 μl of 5× compound that were diluted 100× in solvent if the compound was originally in DMSO. The assay was run such that the maximum tolerable DMSO concentration was 1%. The assay was run at room temperature for 180 minutes. The reference agonist was BDNF. The results are shown in FIG. 13.

### Example 8

### 5-HT7 Human Serotonin GPCR Cell Based Agonist CAMP Assay

This example was conducted to evaluate of the potency (EC50) and efficacy (Max response) of compounds for the human 5-HT7 receptor in stably transfected DLD1 cells determined in a GPCR cell-based cAMP assay.

To start, cells were seeded in a total volume of 15 μL into white walled, 384-well microplates and incubated at 37° C. overnight prior to testing. Prior to testing cell plating media was exchanged with 15 μL of Assay buffer (HBSS+10 mM HEPES). Briefly, intermediate dilution of sample stocks was performed to generate 4× sample in assay buffer. 5 μL of 4× sample was added to cells and incubated at 37° C. for 30 minutes. Final assay vehicle concentration was 1%. The results are expressed as a percent efficacy relative to the maximum response of the control ligand.

The assay volume was 20 μl in a 384-well plate. The compound was added in an amount of 5 μl of 4× compound that were diluted 100× in solvent if the compound was originally in DMSO. The assay was run such that the maximum tolerable DMSO concentration was 1%. The assay was run at 37° C. for 30 minutes. The reference agonist was Serotonin/5-HT.

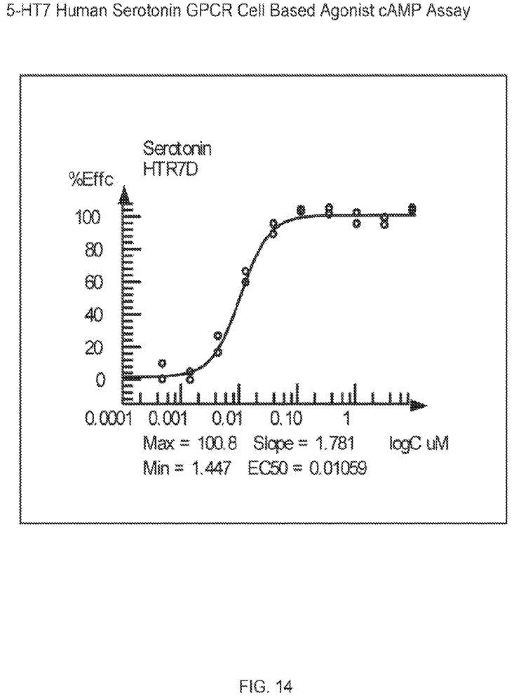

The results are shown in FIG. 14. The amino acid sequence of the 5-HT7 receptor external protein is SEQ ID NO: 2:

```
Amino-acid sequence (extracellular portions 1-83,
139-157, 223-236, 347-367 → PDZ domain
possible at or in between all external sites):
                10 20 30 40
MMDVNSSGRP DLYGHLRSFL LPEVGRGLPD LSPDGGADPV
                50 60 70 80
AGSWAPHLLS EVTASPAPTW DAPPDNASGC GEQINYGRVE
                90 100 110 120
KVVIGSILTL ITLLTIAGNC LVVISVCFVK KLRQPSNYLI
              130 140 150 160
VSLALADLSV AVAVMPFVSV TDLIGGKWIF GHFFCNVFIA
              170 180 190 200
MDVMCCTASI MTLCVISIDR YLGITRPLTY PVRQNGKCMA
              210 220 230 240
KMILSVWLLS ASITLPPLFG WAQNVNDDKV CLISQDFGYT
              250 260 270 280
IYSTAVAFYI PMSVMLFMYY QIYKAARKSA AKHKFPGFPR
              290 300 310 320
VEPDSVIALN GIVKLQKEVE ECANLSRLLK HERKNISIFK
              330 340 350 360
REQKAATTLG IIVGAFTVCW LPFFLLSTAR PFICGTSCSC
              370 380 390 400
IPLWVERTFL WLGYANSLIN PFIYAFFNRD LRTTYRSLLQ
              410 420 430 440
CQYRNINRKL SAAGMHEALK LAERPERPEF VLRACTRRVL
              450 460 470
LRPEKRPPVS VWVLQSPDHH NWLADKMLTT VEKKVMIHD
```

### Example 9

### 5-HT2B Human Serotonin GPCR Cell Based Agonist Calcium Flux Assay

This example was conducted to evaluate of the potency (EC50) and efficacy (Max response) of compounds for the human 5-HT2B receptor in stably transfected HEK293 cells. Assay principle is Calcium Mobilization based assay.

To start, cells were seeded in a total volume of 20 μL into white walled, 384-well microplates and incubated at 37° C. overnight. Prior to testing cell plating media was exchanged with 20 μL of Dye Loading buffer (HBSS+20 mM HEPES containing 1× Dye, 1× Additive A and 2.5 mM Probenecid). Plates were incubated at 37° C. for 45 mins followed by 15 mins at room temperature.

10 μl of assay buffer (HBSS+20 mM HEPES) was added to cells. Intermediate dilution of sample stocks was performed to generate 4× sample in assay buffer. Assay plates, compound plates were loaded into FLIPR instrument. 10 μL of sample was added using FLIPR onboard robotics after 5 seconds of starting Calcium measurement. Final assay vehicle concentration was 1%. The results are expressed as a percent efficacy relative to the maximum response of the control ligand.

The assay volume was 30 μl in a 384-well plate. The compound was added in an amount of 10 μl of 4× compound that were diluted 100× in solvent if the compound was originally in DMSO. The assay was run such that the maximum tolerable DMSO concentration was 1%. The assay was run at 37° C. and the results were read as the compounds were added. The reference agonist was Serotonin.

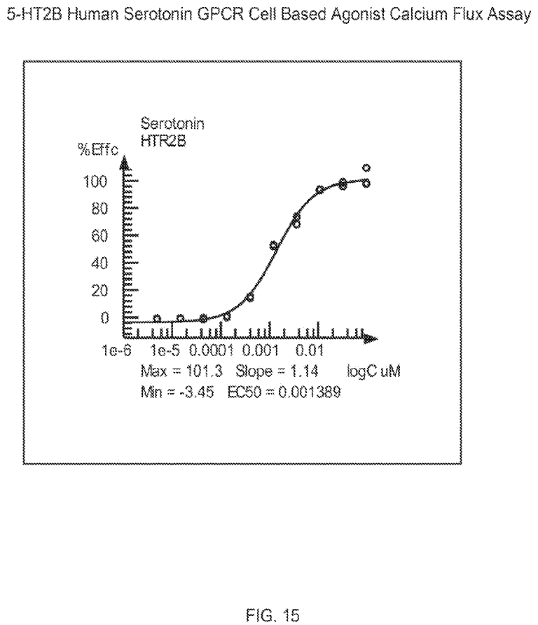

The results are shown in FIG. 15. The amino acid sequence of the 5-HT2A receptor external protein is SEQ ID NO: 3:

```
5-HT2B receptor external protein
Amino-acid sequence (extracellular portion 1-56,
114-129, 193-216, 346-360):
                10 20 30 40
MALSYRVSEL QSTIPEHILQ STFVHVISSN WSGLQTESIP
                50 60 70 80
EEMKQIVEEQ GNKLHWAALL ILMVIIPTIG GNTLVILAVS
                90 100 110 120
LEKKLQYATN YFLMSLAVAD LLVGLFVMPI ALLTIMFEAM
              130 140 150 160
WPLPLVLCPA WLFLDVLFST ASIMHLCAIS VDRYIAIKKP
              170 180 190 200
IQANQYNSRA TAFIKITVVW LISIGIAIPV PIKGIETDVD
              210 220 230 240
NPNNITCVLT KERFGDFMLF GSLAAFFTPL AIMIVTYFLT
              250 260 270 280
IHALQKKAYL VKNKPPQRLT WLTVSTVFQR DETPCSSPEK
              290 300 310 320
VAMLDGSRKD KALPNSGDET LMRRTSTIGK KSVQTISNEQ
              330 340 350 360
RASKVLGIVF FLFLLMWCPF FITNITLVLC DSCNQTTLQM
              370 380 390 400
LLEIFVWIGY VSSGVNPLVY TLFNKTFRDA FGRYITCNYR
              410 420 430 440
ATKSVKTLRK RSSKIYFRNP MAENSKFFKK HGIRNGINPA
              450 460 470 480
MYQSPMRLRS STIQSSSIIL LDTLLLTENE GDKTEEQVSY
V
```

### Example 10

### Perturbed Transcriptional Assay

The assay consists of neuronal cells tested at three phases: 1 baseline healthy cell, 2 perturbed by narcotic, 3 treated with therapeutic lead n, n+1, etc.

Plate n
Grow Neurons with standard expediting procedures including HGH
Stop HGH administration and establish equilibrium
Sample 100 ug or 1/3 of cells cells, mark down barcode on sample tube
place in RNA later solution,
store at −20 C.
Stimulate remaining cells with Narcotic for 48 hours in 27 C. incubation
Sample 100 ug or 1/2 of the remaining cells, mark down
barcode on sample tube
place in RNA later solution,
store at −20 C.
Wash cells
Treat with Therapeutic target for 24 hours in 37 C. incubation
Harvest remaining cells, mark down barcode on sample tube
place in RNA later solution,
store at −20 C.
Control plate
Grow Neurons with standard expiditing procedure including HGH
Stop HGH administration and establish equilibrium
Sample 1/3 or 100 ug minimum of cells, mark down
barcode on sample tube
place in RNA later solution,
store at −20 C.

Stimulate remaining cells with Narcotic for 48 hours in 37C incubation. Then sample minimum of 100 μg or ½ of the cells, mark down barcode on sample tube. Place in RNA later solution and store at −20° C. Next, wash cells, incubate the cells for 24 hours at 37° C. Sample 100 μg cells, mark down barcode on sample tube and place in an RNA later solution and store at −20° C.

### Example 11

### In-Silico Structure Screening-5HT Receptor Binding Candidates

A scoring set of 5 HT receptor binding molecules is being created to evaluate A1 output to guide synthetic approaches including pro-drug like activity. This A1 scoring will then be compared to Docking and activity to identify molecules having the most promise for synthesis as either Pro-drugs, fragments or full drug candidates.

### Autodock

The purpose of using Autodock is to generate a relationship between compound activity to a structure—which can then be used design a novel drug candidates by evaluating those structures in-silico. The quality of the in-silico models can then be compared to Biacore assay, and activity to understand the relationship between binding affinity and agonist/antagonist activity.

A screen might include docking of molecules into various 5H1 crystal structures to generate a set of numerical scores. Such structures could include both agonist and antagonist bound molecules of the same protein.

The primary targets are:
5-HT2A
5-HT2B
5-HT7
TRKB

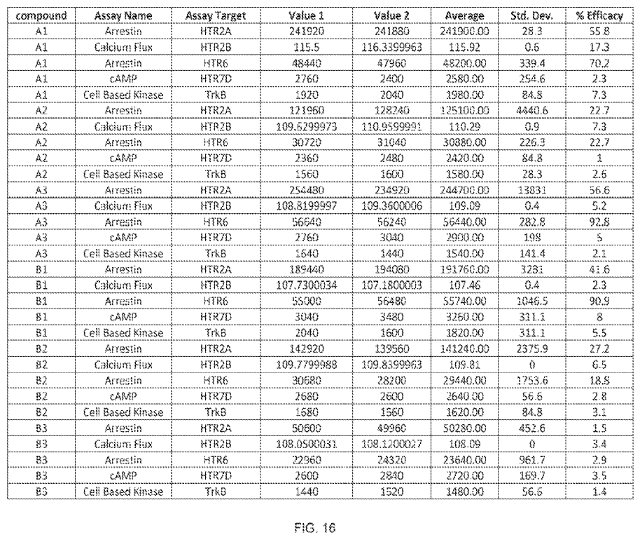

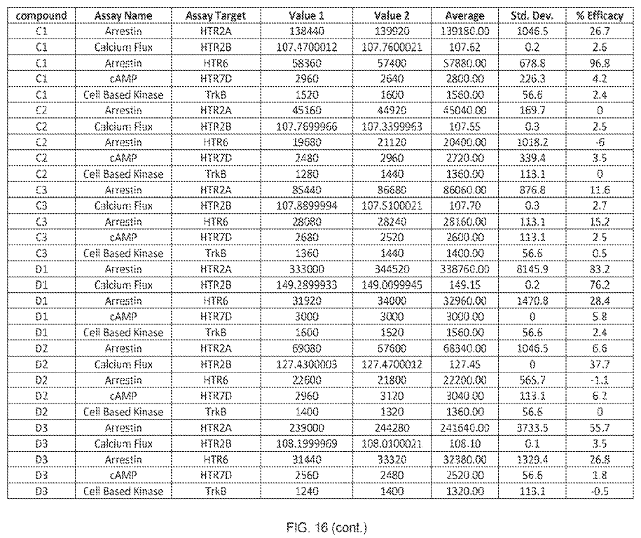

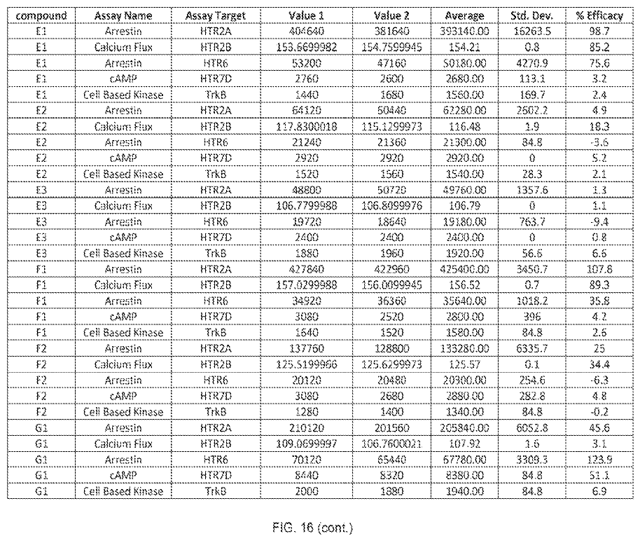

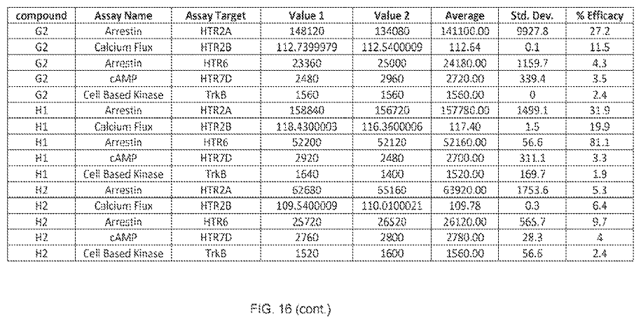

FIG. 16 is a table of the results of agonist assays from each molecule: A1, B1, C1, D1, E1, F1, G1, H1, A2, B2, C2, D2, E2, F2, G2, H2, A3, B3, C3, D3 and E3. Each molecule was tested at a concentration of 10 μM. As shown, the molecules were tested in each assay: Arrestin, Calcium Flux, Arrestin, CAMP, Cell Based Kinase. The targets were HTR2A, HTR2B, HTR6, HTR7D and TrkB respectively.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Sequence Listings
(5-HT2A receptor external protein)
SEQ ID NO: 1
MDILCEENTS LSSTTNSLMQ LNDDTRLYSN DFNSGEANTS
DAFNWTVDSE NRTNLSCEGC LSPSCLSLLH LQEKNWSALL
TAVVIILTIA GNILVIMAVS LEKKLQNATN YFLMSLAIAD
MLLGFLVMPV SMLTILYGYR WPLPSKLCAV WIYLDVLFST
ASIMHLCAIS LDRYVAIQNP IHHSRFNSRT KAFLKIIAVW
TISVGISMPI PVFGLQDDSK VFKEGSCLLA DDNFVLIGSF
VSFFIPLTIM VITYFLTIKS LQKEATLCVS DLGTRAKLAS
FSFLPQSSLS SEKLFQRSIH REPGSYTGRR TMQSISNEQK
ACKVLGIVFF LFVVMWCPFF ITNIMAVICK ESCNEDVIGA
LLNVFVWIGY LSSAVNPLVY TLFNKTYRSA FSRYIQCQYK
ENKKPLQLIL VNTIPALAYK SSQLQMGQKK NSKQDAKTTD
NDCSMVALGK QHSEEASKDN SDGVNEKVSC V
(5-HT7 receptor external protein)
SEQ ID NO: 2
MMDVNSSGRP DLYGHLRSFL LPEVGRGLPD LSPDGGADPV
AGSWAPHLLS EVTASPAPTW DAPPDNASGC GEQINYGRVE
KVVIGSILTL ITLLTIAGNC LVVISVCFVK KLRQPSNYLI
VSLALADLSV AVAVMPFVSV TDLIGGKWIF GHFFCNVFIA
MDVMCCTASI MTLCVISIDR YLGITRPLTY PVRQNGKCMA
KMILSVWLLS ASITLPPLFG WAQNVNDDKV CLISQDFGYT
IYSTAVAFYI PMSVMLFMYY QIYKAARKSA AKHKFPGFPR
VEPDSVIALN GIVKLQKEVE ECANLSRLLK HERKNISIFK
REQKAATTLG IIVGAFTVCW LPFFLLSTAR PFICGTSCSC
IPLWVERTFL WLGYANSLIN PFIYAFFNRD LRTTYRSLLQ
CQYRNINRKL SAAGMHEALK LAERPERPEF VLRACTRRVL
LRPEKRPPVS VWVLQSPDHH NWLADKMLTT VEKKVMIHD
(5-HT2B receptor external protein)
SEQ ID NO: 3
MALSYRVSEL QSTIPEHILQ STFVHVISSN WSGLQTESIP
EEMKQIVEEQ GNKLHWAALL ILMVIIPTIG GNTLVILAVS
LEKKLQYATN YFLMSLAVAD LLVGLFVMPI ALLTIMFEAM
WPLPLVLCPA WLFLDVLFST ASIMHLCAIS VDRYIAIKKP
IQANQYNSRA TAFIKITVVW LISIGIAIPV PIKGIETDVD
NPNNITCVLT KERFGDFMLF GSLAAFFTPL AIMIVTYFLT
IHALQKKAYL VKNKPPQRLT WLTVSTVFQR DETPCSSPEK
VAMLDGSRKD KALPNSGDET LMRRTSTIGK KSVQTISNEQ
RASKVLGIVF FLFLLMWCPF FITNITLVLC DSCNQTTLQM
LLEIFVWIGY VSSGVNPLVY TLFNKTFRDA FGRYITCNYR
ATKSVKTLRK RSSKIYFRNP MAENSKFFKK HGIRNGINPA
MYQSPMRLRS STIQSSSIIL LDTLLLTENE GDKTEEQVSY
V

1. A compound of Formula A1, B1, C1, D1, E1, F1, G1, H1, A2, B2, C2, D2, E2, F2, G2, H2, A3, B3, C3, D3 and E3, or an analog thereof:

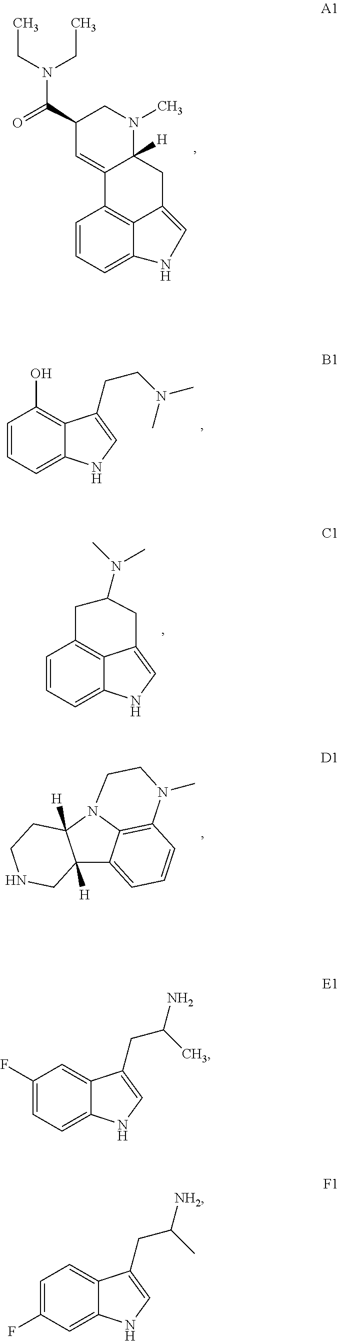 [SMILES: CCN(CC)C(=O)[C@@H]1C=C2c3cccc4[nH]cc(c34)C[C@H]2N(C)C1 ; CN(C)CCc1c[nH]c2cccc(O)c12 ; CN(C)C1Cc2cccc3[nH]cc(c23)C1 ; CN1CCN2c3c(cccc31)[C@@H]1CNCC[C@@H]12 ; CC(N)Cc1c[nH]c2ccc(F)cc12 ; CC(N)Cc1c[nH]c2cc(F)ccc12]

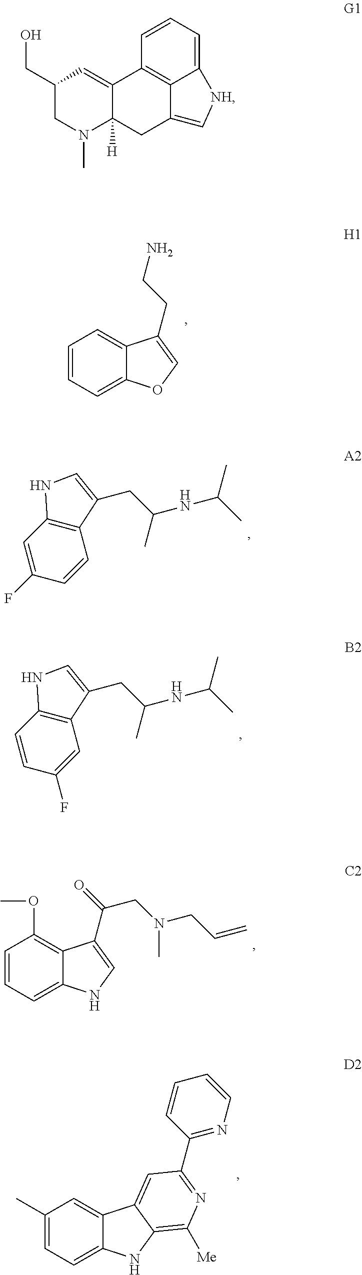

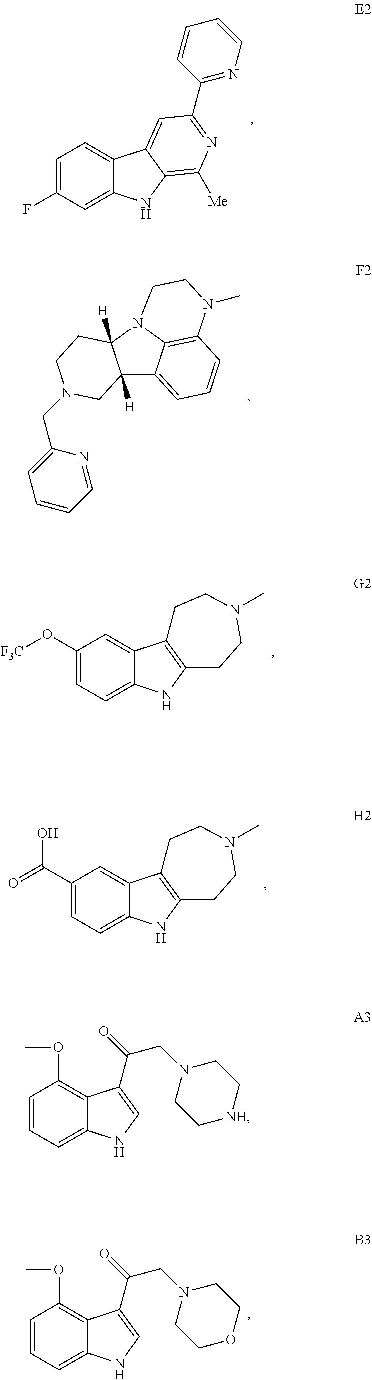

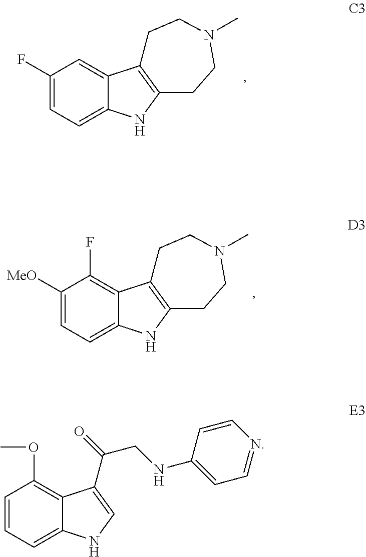

2. A method of treating an ailment comprising administration of an effective amount of the compound of Formula A1, B1, C1, D1, E1, F1, G1, H1, A2, B2, C2, D2, E2, F2, G2, H2, A3, B3, C3, D3, E3 or an analog thereof.

3. The method of claim 2, wherein the ailment is one or more of anxiety, post-traumatic stress disorder (PTSD) and depression.

4. The method of claim 2, wherein the ailment is a neurological disorder.

5. The method of claim 4, wherein the neurological disorder is one or more of acute spinal cord injury, Alzheimer's disease, Amyotrophic Lateral Sclerosis (ALS), ataxia, Bell's Palsy, brain tumors, cerebral aneurysm, epilepsy and seizures, Guillain-Barré Syndrome, headache, head injury, hydrocephalus, meningitis, multiple sclerosis, muscular dystrophy, neurocutaneous syndromes, Parkinson's disease, stroke, headaches, encephalitis and myasthenia gravis.

6. The method of claim 2, wherein the effective amount is between 4 mg and 80 mg.

7. The method of claim 2, wherein the effective amount is between 4 mg and 1.2 grams.

8. A method of increasing neuroplasticity or improving neural function to treat an ailment in a subject in need thereof, the method comprising administering of the compound of A1, B1, C1, D1, E1, F1, G1, H1, A2, B2, C2, D2, E2, F2, G2, H2, A3, B3, C3, D3, E3 or an analog thereof.

9. The method of claim 8, wherein the ailment is one or more of inflammation, addiction, post-traumatic stress disorder (PTSD), traumatic brain injury, depression, acute spinal cord injury, Alzheimer's disease, Amyotrophic Lateral Sclerosis (ALS), ataxia, Bell's Palsy, brain tumors, cerebral aneurysm, epilepsy and seizures, Guillain-Barré Syndrome, headache, head injury, hydrocephalus, meningitis, multiple sclerosis, muscular dystrophy, neurocutaneous syndromes, Parkinson's disease, stroke, headaches, encephalitis and myasthenia gravis.

10.-15. (canceled)

16. The method of claim 2, wherein the ailment is inflammation.

17.-22. (canceled)


SEQUENCE LISTING (3 sequences)

SEQ ID NO: 1 - protein, 471 aa, Homo sapiens
MDILCEENTSLSSTTNSLMQLNDDTRLYSNDFNSGEANTSDAFNWTVDSENRTNLSCEGCLSPSCLSLLHLQEKNWSALLTAVVIILTIAGNILVIMAVSLEKKLQNATNYFLMSLAIADMLLGFLVMPVSMLTILYGYRWPLPSKLCAVWIYLDVLFSTASIMHLCAISLDRYVAIQNPIHHSRFNSRTKAFLKIIAVWTISVGISMPIPVFGLQDDSKVFKEGSCLLADDNFVLIGSFVSFFIPLTIMVITYFLTIKSLQKEATLCVSDLGTRAKLASFSFLPQSSLSSEKLFQRSIHREPGSYTGRRTMQSISNEQKACKVLGIVFFLFVVMWCPFFITNIMAVICKESCNEDVIGALLNVFVWIGYLSSAVNPLVYTLFNKTYRSAFSRYIQCQYKENKKPLQLILVNTIPALAYKSSQLQMGQKKNSKQDAKTTDNDCSMVALGKQHSEEASKDNSDGVNEKVSCV

SEQ ID NO: 2 - protein, 479 aa, Homo sapiens
MMDVNSSGRPDLYGHLRSFLLPEVGRGLPDLSPDGGADPVAGSWAPHLLSEVTASPAPTWDAPPDNASGCGEQINYGRVEKVVIGSILTLITLLTIAGNCLVVISVCFVKKLRQPSNYLIVSLALADLSVAVAVMPFVSVTDLIGGKWIFGHFFCNVFIAMDVMCCTASIMTLCVISIDRYLGITRPLTYPVRQNGKCMAKMILSVWLLSASITLPPLFGWAQNVNDDKVCLISQDFGYTIYSTAVAFYIPMSVMLFMYYQIYKAARKSAAKHKFPGFPRVEPDSVIALNGIVKLQKEVEECANLSRLLKHERKNISIFKREQKAATTLGIIVGAFTVCWLPFFLLSTARPFICGTSCSCIPLWVERTFLWLGYANSLINPFIYAFFNRDLRTTYRSLLQCQYRNINRKLSAAGMHEALKLAERPERPEFVLRACTRRVLLRPEKRPPVSVWVLQSPDHHNWLADKMLTTVEKKVMIHD

SEQ ID NO: 3 - protein, 481 aa, Homo sapiens
MALSYRVSELQSTIPEHILQSTFVHVISSNWSGLQTESIPEEMKQIVEEQGNKLHWAALLILMVIIPTIGGNTLVILAVSLEKKLQYATNYFLMSLAVADLLVGLFVMPIALLTIMFEAMWPLPLVLCPAWLFLDVLFSTASIMHLCAISVDRYIAIKKPIQANQYNSRATAFIKITVVWLISIGIAIPVPIKGIETDVDNPNNITCVLTKERFGDFMLFGSLAAFFTPLAIMIVTYFLTIHALQKKAYLVKNKPPQRLTWLTVSTVFQRDETPCSSPEKVAMLDGSRKDKALPNSGDETLMRRTSTIGKKSVQTISNEQRASKVLGIVFFLFLLMWCPFFITNITLVLCDSCNQTTLQMLLEIFVWIGYVSSGVNPLVYTLFNKTFRDAFGRYITCNYRATKSVKTLRKRSSKIYFRNPMAENSKFFKKHGIRNGINPAMYQSPMRLRSSTIQSSSIILLDTLLLTENEGDKTEEQVSYV